(19) United States
(12) Patent Application Publication
(10) Pub. No.: US 2024/0189539 A1
(43) Pub. Date: Jun. 13, 2024

(54) Sensory Stimulation Therapy Device Using LCD Sunglasses

(71) Applicant: Tomoo MATSUDA, Fujisawa (JP)

(72) Inventor: Tomoo MATSUDA, Fujisawa (JP)

(21) Appl. No.: 18/185,418

(22) Filed: Mar. 17, 2023

Related U.S. Application Data
(60) Provisional application No. 63/432,163, filed on Dec. 13, 2022.

(51) Int. Cl. A61M 21/00; G02C 7/10

(52) U.S. Cl. CPC A61M 21/00; G02C 7/101; A61M 2021/0027; A61M 2021/0044; A61M 2205/33; A61M 2209/088

(57) ABSTRACT

A sensory stimulation therapy device for improving functional connectivity of brain to prevent and treat neurological disorders, using LCD sunglasses enabling light and sound entrainment combined with exercise and cognitive therapies, is disclosed. To handle an LCD element with slow response, light stimulation is generated with continuous light with a smooth waveform rather than pulsed light with a steep waveform. Variations in therapeutic effects are also prevented by stably controlling a phase difference Δθ between a fundamental frequency component of the light stimulation and sound stimulation generation timing to a desired value.

### CROSS REFERENCE

The present application claims priority under 35 U.S.C. § 119 (e) to U.S. Provisional Patent Application No. 63/432,163, titled SENSORY STIMULATION THERAPY DEVICE USING LCD SUNGLASSES, filed on Dec. 13, 2022, which is expressly incorporated by reference in its entirety.

### BACKGROUND OF THE INVENTION

### Field of the Invention

The present invention relates to a sensory stimulation therapy device for improving functional connectivity of brain to prevent and treat neurological disorders, using LCD sunglasses having features enabling a subject of treatment to see the surrounding environment and combining light and sound entrainment with exercise and cognitive therapies.

### Description of the Related Art

In the related art, Gamma ENtrainment Using Sensory stimuli (GENUS) performs “light and sound entrainment” for inducing therapeutic brain waves through visual stimulation by generating pulsed light with a steep waveform from an LED emitter flashing in the dark and through sound stimulation by emitting pulsed sound from an audible-frequency sound source. The phase difference of the pulse waveforms of the light and sound stimulation ranges from −180 degrees to +180 degrees.

A report from a phase II clinical trial in 2021 using a stationary therapeutic device showed excellent trial results, such as no serious adverse events, reduced atrophy of Alzheimer's ventricles, and improved associative memory of names and faces. However, no significant improvement in performance on multiple cognitive tests was reported.

Current reports indicate that a portable therapeutic device having a member like an eye mask worn on the head to block out ambient light and cause the subject of treatment to stare at pulsed light generated by an LED emitter in the dark has received a Breakthrough Device Designation (BDD) from the US FDA in 2021, and is being prepared for a large-scale phase III clinical trial.

Patients with Alzheimer's disease and mild cognitive impairment (MCI) and their families around the world have high expectations for GENUS clinical research, which aims for development as digital medicine.

Many studies have been conducted on each of “light entrainment” and “sound entrainment” in the related art as standalone phenomena, using various frequencies and waveform patterns. For steady-state visually evoked potentials (SSVEP) that stimulate vision with light, the phenomenon of entrainment, in which brain waves are induced by light in the frequency range from 1 Hz to 90 Hz and higher, is well known. Within this frequency range, entrainment occurs in response to one or more fundamental and harmonic frequencies in the waveform pattern of the light stimulation. The auditory steady-state response test (ASSR) that stimulates hearing with pulsed (clicking) or continuous (chirping) sounds as sound stimulation, is also well known as a phenomenon in which entrainment occurs in correspondence with one or more repeating frequencies.

Regarding the “effects of phase difference when phase-synchronized stimulation is applied to two locations in the brain” in the related art, the results of a test applying stimulation to two locations at the front and side of head were published in 2019. The results show that when stimuli of opposite phase (phase difference ±180 degrees) are applied to two locations, a temporary cognitive impairment occurs in healthy subjects.

Similarly, light stimulation generates brain waves in the back of head with SSVEP, and sound stimulation generates brain waves in the top of head with ASSR. If “light entrainment” and “sound entrainment” are phase-synchronized to simultaneously stimulate two locations in the brain, brain waves are generated at the two locations for each stimulus. Thus, some say that “applying an inappropriate phase difference between light and sound stimulation can cause brain waves generated separately at the two locations to interfere with or cancel each other out, potentially disrupting the brain waves. Caution should be exercised for adverse events such as headaches.” Currently, however, the specific conditions that produce adverse events are unknown.

Regarding the “therapeutic effects of improving functional connectivity of brain to treat various neurological disorders” in the related art, the triple network model was proposed in 2011 to investigate neurological disorders as functional disorders of the brain network. One document (WO2013/152348) providing frequency coupling to elements of the body, including the brain, to treat various neurological disorders proposes the idea of providing a patient with auditory and visual stimuli that repeat on the frequency of brain waves. According to the patent publication of this document submitted to Japan (JP2015-519096A), Alzheimer's disease is also targeted for treatment, and claim 10 indicates that gamma brain wave (Δθ Hz) frequencies are included.

Recently, many other examples of “research into improving functional connectivity of brain to treat various neurological disorders” have been reported.

In the related art, GENUS described above is expected to be useful in the treatment of Alzheimer's disease based on the mechanism of action to reduce amyloid-β and tau proteins since the disclosure of successful animal experiments in 2016.

Simultaneously, GENUS has made great achievements in confirming the “safety of Δθ Hz light and sound entrainment” as a result of clinical research. This is because if entrainment at Δθ Hz is safe for treatment, then medical researchers can safely address various neurological disorders caused by brain network dysfunction by using gamma wave stimulation around about Δθ Hz to study entrainment treatments that increase functional connectivity.

A health device that relaxes with alpha wave light stimulation around about 10 Hz is already commercially available and used by many users. Thus, the safety and effectiveness of entrainment at 10 Hz and Δθ Hz in combination and at other frequencies are expected to be confirmed in future clinical research.

To apply light and sound entrainment to various clinical research in the future, the availability of a therapeutic device for clinical research that supports frequencies other than Δθ Hz is desirable.

As another technology of the related art, efforts are underway to develop several types of “transparent glasses with LED emitters” (for example, US2018/0185665A1 and WO2018/094226). If successful, the subject of treatment will be able to see the surrounding environment, thereby enabling exercise and cognitive therapies combined with GENUS.

However, it should be noted that for exercise therapy that includes walking outdoors, the illuminance can exceed 100,000 lux by the sea and in the mountains in summer, and the illuminance can exceed 10,000 lux on sunny days even in parks and hospital grounds in urban areas. For example, if a patient is taking a walk in the sunshine on a sunny day and enters a dark area in the shade of a tree or a building, the surroundings will darken suddenly. The pupil of the eye exposed to light stimulation from an LED emitter that flashes brighter than the ambient daylight is also exposed to intense light from the LED emitter at the instant of entering the shade. As a result, the pupil of a patient who enters a dark shaded area while walking may be unable to open quickly and responsively enough, making the patient feel the surroundings darker, and furthermore, the patient may be at risk of falling.

Conversely, with cognitive therapy performed seated in a chair, there is little risk of falling due to a flashing emitter. However, there is a possible risk of impairing cognitive activity in cognitive therapy if intense pulsed light is received from an LED emitter in front of the eyes while performing cognitive therapy by reading figures and characters in indoor lighting of around 1,000 to 300 lux.

Either way, the GENUS portable therapy device that has received BDD from the FDA currently adopts the above-described “eye mask with LED emitters”, which is difficult to use together with exercise and cognitive therapies. At least at present, there are difficulties with the technological development and the like of “transparent glasses with LED emitters”.

In the related art, US2020/0108270A1 discloses an invention aiming to apply light and sound entrainment as “LCD sunglasses that switch between only the two states of transparent and opaque”. According to this document, LCD sunglasses of this type are an adaptation of proven LCD sunglasses for visual training of astronauts and the like.

Taking advantage of how “automatic brightness regulation by the pupil of the human eye” is not impaired excessively, LCD sunglasses have also been applied to sports equipment to train the dynamic vision of athletes, and are commercially available for several hundred dollars. This commercial product has a function of repeatedly transmitting/blocking ambient light at a freely set frequency, and has been used in many cases to train an athlete's ability to see a ball flying at high speed in sports such as tennis and baseball.

However, none of the documents cited above discloses how to set the phase difference Δθ between light and sound stimulation to prevent variations in therapeutic effects and adverse events in treatment by light and sound entrainment using LCD sunglasses.

### SUMMARY OF THE INVENTION

Accordingly, first, to combine exercise and cognitive therapies with treatment by light and sound entrainment, it is desirable to use LCD sunglasses that do not excessively impair the automatic brightness regulation by the pupil of the human eye.

Second, to prevent variations in therapeutic effects and adverse events in light and sound entrainment using LCD sunglasses, a technique for properly setting the phase difference Δθ between light and sound stimulation is required.

As above, LCD sunglasses have the significant merit of not excessively impairing the automatic brightness regulation by the pupil of the human eye, and are suitable for entrainment by light stimulation while the subject is viewing the surrounding environment. However, there are two issues that need to be resolved to use an LCD element in a sensory stimulation therapy device.

The first issue is that the response and linearity of an LCD element are markedly inferior to a conventional LED emitter, which is unsuitable for generating “pulsed light with a steep waveform”.

Accordingly, in the present invention, entrainment is performed using the phenomenon of SSVEP for the fundamental frequency component of continuous light by using the LCD element to generate “continuous light with a smooth waveform” instead of “pulsed light with a steep waveform”.

The second issue is that the phase difference Δθ between light and sound stimulation during entrainment therapy cannot be achieved at the exact desired value due to waveform distortion and the significantly slower response of the LCD element compared to the LED emitter used in the related art. This results in unstable entrainment and variations in therapeutic effects.

Accordingly, the present invention focuses on the light stimulation onset delay θL due to the poor response of the LCD element. For the LED emitter of the related art, θL is substantially 0, but for the LCD element, the value of θL fluctuates greatly depending on the waveform pattern of the light stimulation.

In the present invention, the numerical value of OL of the LCD element is measured in advance for each waveform pattern of the light stimulation to be used for entrainment. Since θs that indicates the timing for generating the sound stimulation can be calculated by the relation θs=θL+Δθ, θs is calculated by adding the measured θL and desired Δθ values, and the sound stimulation is generated in accordance with the value of θs.

Accordingly, Δθ can be controlled accurately to the desired value, and the problem is solved.

In a preferred embodiment, first, after showing that the LCD element is not suitable for generating “pulsed light with a steep waveform” as in the related art, the principle of using the LCD element to generate continuous light stimulation and achieve entrainment with SSVEP to generate brain waves is described as a solution. Next, the principle of using an audible sound source to generate sound stimulation and achieve entrainment with ASSR is described. Furthermore, the temporal relationship regarding the phase difference among the control start timing (point A), the light stimulation start timing (point B), and the sound stimulation start timing (point C) is described.

Next, after illustrating a block diagram of a feed-forward control system of LCD sunglasses of the present invention, the functions of each control block is explained in detail. Thereafter, a configuration example of means for developing a sensory stimulation therapy device is illustrated, and a technique for measuring θL of the LCD element and an example of measuring θL for each waveform pattern is described.

Finally, an overall control routine for outputting light and sound stimulation which is incorporated into the control device is described in the following order.

First, a control routine of the LCD sunglasses is described. Specifically, waveform data of the absolute value of applied voltage of the LCD element is calculated by first generating waveform data of a transmittance target value of the LCD element and then substituting into an approximation formula of an inverse mapping correction part that calculates a voltage E to be applied from a desired transmittance P. An AC voltage is then generated in a polarity reversal part that reverses the polarity of the applied voltage to apply the voltage to the LCD element.

Second, the pre-measured numerical value of the onset delay θL of light stimulation is used to calculate θs by substituting the measured θL and the desired Δθ into the relation θs=θL+Δθ, and the timing for generating sound stimulation is determined on the basis of θs.

Third, sound stimulation is generated at an appropriate timing calculated as θs.

By repeating the above three processes, it is possible to stabilize the phase difference Δθ between light and sound stimulation using continuous light and achieve a sensory stimulation therapy device using LCD sunglasses.

The preferred embodiment is advantageous in that a method of developing LCD sunglasses that allow viewing of the surrounding environment is disclosed in detail using an example of a combination of readily available equipment, such that light and sound entrainment with LCD sunglasses can be achieved.

As a result, a sensory stimulation therapy device with fewer variations in therapeutic effects can be achieved when using entrainment combined with exercise and cognitive therapies.

### BRIEF DESCRIPTION OF THE DRAWINGS

FIG. 1A is a diagram explaining why an LCD element is unsuitable for entrainment by pulsed light with a steep waveform;

FIG. 1B is a diagram of entrainment with an LCD element by using continuous light to perform SSVEP for vision;

FIG. 1C is a diagram of entrainment with a sound-emitting device by using pulsed sound to perform ASSR for hearing;

FIG. 1D illustrates the positional relationship among a control start point (point A), a light stimulation start point (point B), and a sound stimulation start point (point C); incidentally, the phase difference from point A to B is designated θL, the phase difference from point B to C is Δθ, and the phase difference from point A to C is θs;

FIG. 2 is a configuration diagram of a feed-forward control system of the present invention that generates continuous light with LCD sunglasses;

FIG. 3 illustrates examples of fundamental waveforms usable for generating a transmittance target value 205 in a transmittance target generator 207;

FIG. 4A is a diagram explaining a method of measuring the correspondence relationship between an applied voltage E and a specific transmittance P of an LCD element;

FIG. 4B is a plot depicting an example of measuring the correspondence relationship from the applied voltage E to the specific transmittance P of the LCD element;

FIG. 4C is a plot (mapping graph) depicting the correspondence relationship from the absolute value E of applied voltage to the specific transmittance P of the LCD element;

FIG. 4D is a plot (inverse mapping graph) depicting the correspondence relationship from the specific transmittance P to the absolute value E of applied voltage of the LCD element;

FIG. 5A is an example of an approximation formula for implementing an inverse mapping correction part 237 (inverse mapping graph) in a computer;

FIG. 5B illustrates an example of using readily available electronic components to configure a polarity reversal part 250;

FIG. 6 is a configuration example of an apparatus for system development and quality control of a sensory stimulation therapy device using LCD sunglasses;

FIG. 7 is a diagram illustrating collapse of a transmittance waveform when a square wave is used as a transmittance target, and an example of measuring the light stimulation onset delay (or advance, in some cases) θL;

FIG. 8 is a diagram illustrating delay of a transmittance waveform when a sine wave is used as the transmittance target, and an example of measuring the light stimulation onset delay θL;

FIG. 9A is a control flowchart of a sensory stimulation therapy device using LCD sunglasses;

FIG. 9B is an overview of routine R05 and illustrates a procedure for controlling LCD sunglasses 10 and earphones 168;

FIG. 10A is an overview of routine R051 and illustrates a procedure for controlling the LCD sunglasses 10;

FIG. 10B is a diagram explaining a process related to a phase difference Δθ applied in routine R052;

FIG. 10C is a time series chart of the control of the earphones 168 in routine R053;

FIG. 11A is an example of outdoor exercise therapy;

FIG. 11B is an example of indoor cognitive therapy;

FIG. 11C illustrates alternative embodiments of various outward shapes of LCD sunglasses;

FIG. 11D is a product concept of oversize LCD sunglasses that can be used while wearing glasses 7 for daily use;

FIG. 11E is a conceptual diagram of connecting a sensory stimulation therapy device 20 to a smartphone 302;

FIG. 12A is an alternative embodiment in which stereo audio is played on the smartphone 302 and outputted to an audio-visual control device 50;

FIG. 12B illustrates an example of waveform patterns of audio data for light stimulation 200 and sound stimulation 210 included in stereo audio; and

FIG. 12C is a diagram explaining the structure of the audio-visual control device 50 that demodulates light stimulation 200 and sound stimulation 210 from stereo audio.

### DETAILED DESCRIPTION OF THE PREFERRED EMBODIMENTS

The following description refers to the accompanying drawings which form part of this specification and illustrate several embodiments of the present invention. It is understood that other embodiments can be used, and structural and operational changes can be made, without departing from the scope of the present invention.

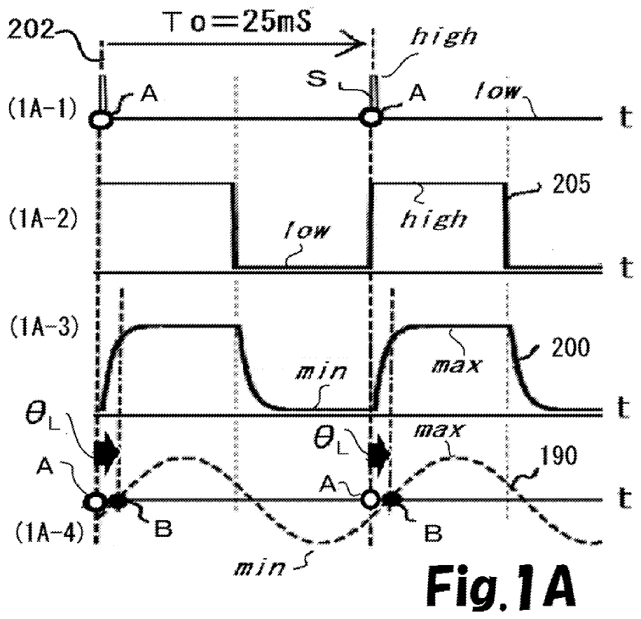

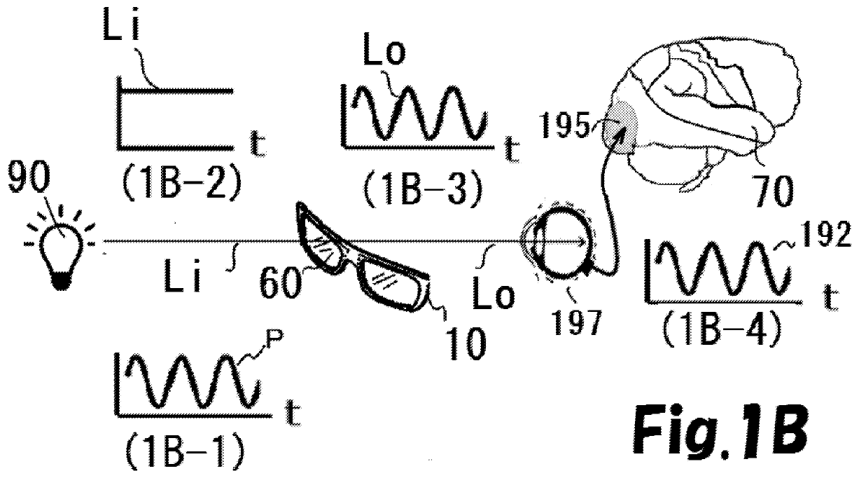

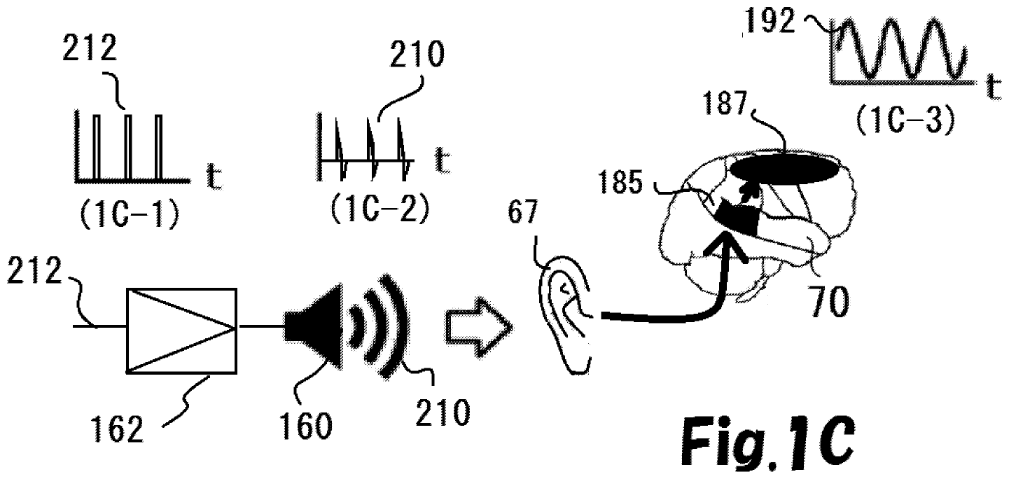

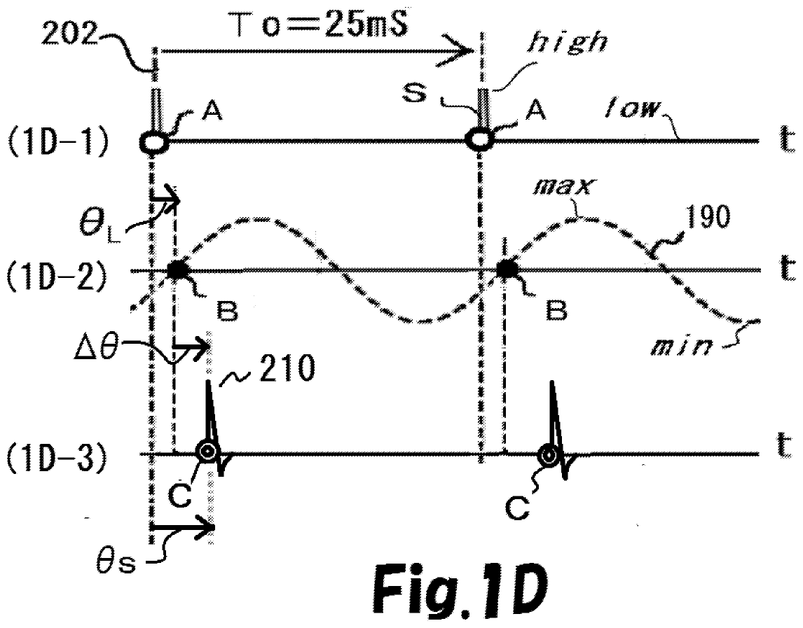

FIG. 1A illustrates why pulsed light with a steep waveform cannot be generated as light stimulation in an LCD element 60.

The uppermost time series graph (1A-1) represents a signal S indicating the timing for repeating a control every cycle T0=25 ms as an example. A timing A (hereinafter designated point A) as the start point 202 of control cycle T0 is denoted with a white circle. The horizontal axis of the time series graph is time t [ms]. The signal S takes a logical value “high” during a first sampling interval that includes point A, and takes a logical value “low” otherwise. The signal S is designated the phase synchronization signal S.

The time series graph (1A-2) second from the top is an example of setting the waveform pattern of a transmittance target value 205 of the LCD element 60 to a square wave with a 50% duty cycle. In the diagram, the square wave only switches between the two logical values “high” and “low”. In other words, the problem with the LCD element is explained using the same waveform pattern as the command method of the related art in which an LED emitter is flashed as pulsed light with a steep waveform.

The time series graph (1A-3) third from the top is the waveform of the brightness of light stimulation 200 by the LCD element 60 produced by the transmittance target value 205 above. If the transmittance target value 205 continues to be the logical value “high”, the waveform of the light stimulation 200 rises to a maximum value “max”. If the transmittance target value 205 continues to be the logical value “low”, the waveform of the light stimulation 200 falls to a minimum value “min”. However, at the instant when the transmittance target value 205 switches between the logical values “high” and “low”, the waveform of the light stimulation 200 “smoothly varies” between the maximum value “max” and the minimum value “min”, and thus the waveform of the light stimulation 200 does not form a square wave as pulsed light with a steep waveform.

In this way, the LCD element 60 cannot output square-wave pulsed light with a steep waveform compared to an LED emitter with good response used in the related art, with which square-wave pulsed light with a steep waveform can be obtained if the target value of the square wave is given.

The time series graph (1A-4) fourth from the top or lowermost illustrates the extracted waveform of a fundamental frequency component 190 included in the light stimulation 200 described above. The waveform of the fundamental frequency component 190 draws a sine wave between the minimum value “min” as the negative value and the maximum value “max” as the positive value. The timing B (hereinafter designated point B) of the zero-crossing point along the way as the waveform of the fundamental frequency component 190 increases from the negative minimum value “min” to the positive maximum value “max” is denoted with a black circle.

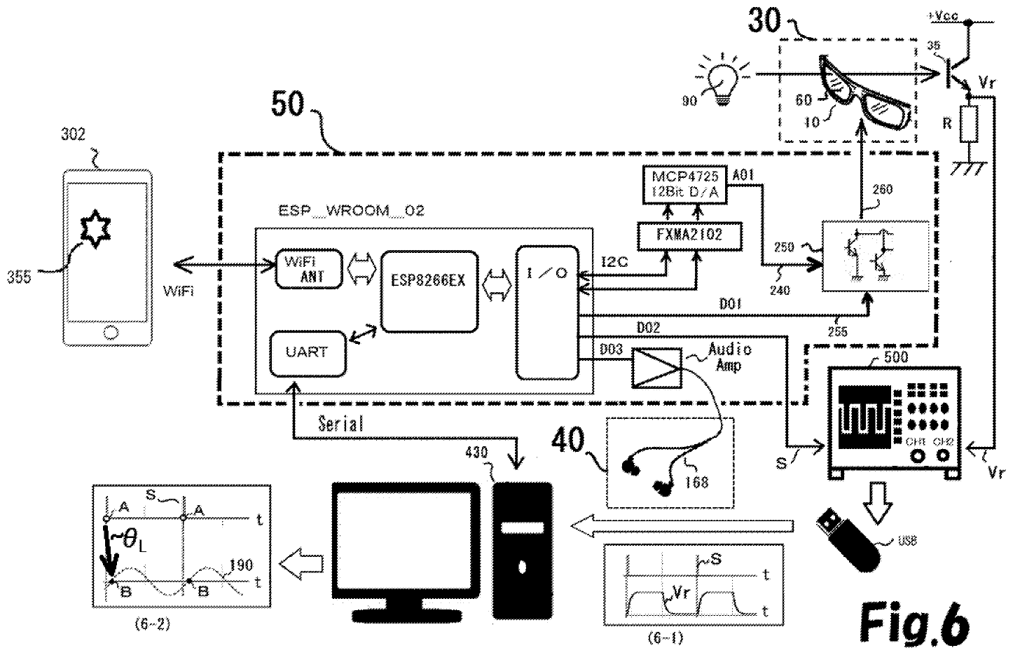

A specific procedure for measuring the “phase delay OL from points A to B” illustrated in FIG. 1A is described. See FIG. 6 for a description of the configuration of the measuring means.

First, the two-channel signal of the phase synchronization signal S of the time series graph (1A-1) and a voltage Vr measured by a semiconductor illuminance sensor 35 such as a phototransistor NJL7502L to measure the brightness of the light stimulation 200 of the time series graph (1A-3) are synchronized while data is collected by a digital oscilloscope (for example, IWATSU-DS5105B) equipped with a high-speed data logger function.

Next, the collected digital data is passed to MATLAB(R) on a PC via USB memory, and the FFT and inverse FFT are applied in MATLAB (R) to obtain data of the fundamental frequency component 190. The data of the fundamental frequency component 190 is obtained by simply applying the FFT transform to the digital data of the voltage Vr in the numerical analysis software MATLAB (R), zero-clearing the data other than the target fundamental frequency component 190 from the complex data of the FFT result, and then applying the “inverse FFT transform”. The result of the “inverse FFT transform”, when plotted on a time series graph, is the waveform pattern of the fundamental frequency component 190 of the voltage Vr.

Finally, the phase synchronization signal S and the fundamental frequency component 190 of the voltage Vr are plotted on a time series graph. The “phase delay θL from points A to B” can be measured by comparing, in MATLAB (R), the timings of point A at which the phase synchronization signal S rises and point B of the zero-crossing as the sine wave of the fundamental frequency component 190 of the voltage Vr increases.

The configuration of the above measuring means is also described later in FIG. 6.

From the perspective of the start point 202 of the timing for repeating control, the “phase delay θL from points A to B” can also mean the delay θL until the light stimulation is generated. The description below is from this perspective.

Point A denoted by the white circle in the uppermost time series graph (1A-1) is also displayed as point A with a white circle at the same timing in the lowermost time series graph (1A-4). Although the transmittance target value 205 is a square wave with a steep waveform in the time series graph (1A-2), the waveform of the light stimulation 200 in the time series graph (1A-3) collapses because of the poor response of the LCD element. As a result, as displayed in the time series graph (1A-4), point B drawn with a black circle occurs later than point A drawn with a white circle. The phase delay from points A to B is designated θL hereinafter.

Since the LED emitter used in the related art has fast response, θL=0 at all times, with no delay between points A and B, and when designating the phase difference Δθ between light and sound in the related art, the starting point of the light stimulation is always point A.

Meanwhile, in the LCD element with slow response, the “phase delay θL from points A to B” exists, and thus the starting point of light stimulation in SSVEP is not point A, and instead point B shifted by the phase difference θL is the SSVEP starting point.

In LCD sunglasses predating the present invention, the existence of θL was overlooked. After investigating the cause of the problem in detail, the inventor discovered that this neglected θL is the cause of the problem of not being able to accurately manage and achieve a desired value for the phase difference Δθ between light and sound.

FIG. 1B is a diagram explaining the principle of LCD sunglasses 10 that entrain the fundamental frequency component 190 of continuous light in the back 195 of the brain using the phenomenon of SSVEP in which the waveform pattern of the fundamental frequency component 190 of light stimulation generates brain waves.

Light emitted from a light source 90 that shines at constant brightness becomes incident light Li, and upon passing through the LCD element 60 of LCD sunglasses 10, the brightness of transmitted light Lo also changes with a waveform pattern including a component similar to the waveform pattern in which the specific transmittance P changes. Incidentally, the transmittance of LCD sunglasses cannot be lowered to 0 to create complete darkness. Consequently, a portion of the incident light Li acting as ambient light is mixed with the transmitted light Lo to form continuous light. This continuous light reaches an eyeball 197 and stimulates the optic nerve, generating a brain wave 192 in the primary visual cortex 195 of the brain 70. In the phenomenon of SSVEP, entrainment by light stimulation occurs by inducing a brain wave 192 corresponding to the frequency component approximately ranging from 1 Hz to 90 Hz in visual stimulation.

The time series graph (1B-1) in FIG. 1B illustrates an example of a waveform pattern resulting from controlling the specific transmittance P of the LCD element 60 of the LCD sunglasses 10, and includes a fundamental frequency fn as a sine wave and a harmonic frequency fm at an integer multiple of the fundamental frequency. The fundamental frequency and the harmonic frequency may each be singular or plural frequencies.

In the time series graph (1B-2), the illuminance of the incident light Li incident on the LCD element 60 is measured in units of lux, and since the brightness of the light source 90 is constant, the illuminance of the incident light Li is also constant.

In the time series graph (1B-3), the illuminance of the transmitted light Lo transmitted through the LCD element 60 is measured in units of lux, and since the illuminance of the incident light Li is constant, a waveform pattern similar to the waveform pattern of the specific transmittance P in the time series graph (1B-1) appears. Consequently, the fundamental frequency fn and the harmonic frequency fm are included.

The time series graph (1B-4) schematically illustrates the brain wave 192 of a frequency generated by entrainment in the primary visual cortex 195 of the brain 70 by the phenomenon of SSVEP, and includes the above fundamental frequency in and harmonic frequency fm in the frequency range approximately from 1 Hz to 90 Hz.

As illustrated in FIGS. 1A and 1B, the LCD sunglasses 10 incorporating the LCD element 60 are unsuitable for using pulsed light with a steep waveform because the response of the LCD element 60 is markedly inferior to the LED emitter of the related art.

However, it is known that entrainment of light stimulation occurs by the phenomenon of SSVEP if continuous light including the fundamental frequency fn and the harmonic frequency fm of the fundamental frequency fn is used in the frequency range approximately from 1 Hz to 90 Hz.

Thus, a characteristic of the LCD sunglasses 10 is that the transmittance can be controlled by setting the transmittance target value 205 to generate continuous light containing one or multiple fundamental frequencies fn and harmonic frequencies fm of the fundamental frequency fn.

Furthermore, in the LCD sunglasses of the present invention described below, not only the frequency of the generated light stimulation 200 but also the upper/lower limits, the average, or the waveform pattern of continuous light can be set or adjusted by the transmittance target value 205.

FIG. 1C is a diagram explaining the principle by which entrainment of sound stimulation in the top 187 of the brain 70 occurs using the phenomenon of ASSR in which a repeating frequency of sound supplied to the auditory system is expressed as a brain wave.

If a pulsed sound drive voltage 212 of a square wave as a click sound is inputted into an amplifier 162, pulsed sound stimulation 210 is generated from a sound-emitting device 160 driven by the amplifier. The sound stimulation 210 propagates through the air and reaches an ear 67 of the subject, and a nerve signal travels through the primary auditory cortex 185 and is detected as a brain wave 192 near the top 187 of the head.

The time series graph (1C-1) is a short square wave as a click sound inputted into the amplifier 162. The time series graph (1C-2) is the waveform pattern of pulsed sound stimulation 210 propagating through the air from the sound-emitting device 160. The time series graph (1C-3) is the waveform of the brain wave 192 of a frequency generated by entrainment, which is detected near the top 187 of the head.

FIG. 1D illustrates the positional relationship among the control start point (point A), the light stimulation start point (point B), and a point C as the sound stimulation start point. The control start point (point A) is the timing 202 at the start of the initial sampling interval of the control cycle T0 illustrated in FIG. 1A. The light stimulation start point (point B) is the zero-crossing point as the fundamental frequency component 190 of the light stimulation 200 illustrated in FIG. 1A increases. The sound stimulation start point (point C) is the timing when the waveform of the sound stimulation 210 is generated as sound by the sound-emitting device 160, and is assumed to be substantially equal to the timing when the sound drive voltage 212 is inputted into the amplifier 162 having a negligible time delay.

The time series graph (1D-1) illustrates the phase synchronization signal S that rises the logical value “high” only in the initial sampling interval of the control cycle, being the signal indicating point A denoted by the white circle. The time series graph (1D-2) illustrates the zero-crossing point (point B denoted by the black circle) as the fundamental frequency component 190 of the light stimulation increases. The time series graph (1D-3) illustrates the start point (point C denoted by the double circle) of the control timing for generating the sound stimulation 210.

In these time series graphs, the position of point A indicates the timing when the control cycle T0 starts. The position of point B is influenced by the waveform pattern of the transmittance control of the LCD element 60, and the nonlinearity or delayed response of the LCD element 60. Points B and A occur at different positions, except under special conditions. However, by taking measurements with the device in FIG. 6 described later, the phase difference OL from point A to B can be analyzed and calculated. Point C can be set as the timing for generating sound stimulation by intentionally programming the control device.

Accordingly, the phase delay θL from point A to B is designated the “light stimulation onset delay θL”.

The phase difference Δθ from points B to C is designated the “phase difference Δθ between light and sound stimulation”.

The phase delay θs from points A to C is designated the “timing θs for generating sound stimulation”.

Thus, as illustrated in FIG. 1D, given the positional relationship of points A, B, and C, the relation θs=θL+Δθ always holds. Of these, the “light stimulation onset delay θL” is determined by the characteristics of the LCD element 60 and the waveform pattern, and thus can be measured at the design stage or at the manufacturing and inspection stage.

The “phase difference Δθ between light and sound stimulation” influences the phase difference between the brain wave 192 generated in the back 195 of the head by the light stimulation 200 and the brain wave 192 generated near the top 187 of the head by the sound stimulation 210. Thus, Δθ also affects therapeutic effects and adverse events. Accordingly, it is appropriate to determine a useful value through clinical trials accompanied by medical judgment and then assign the desired value to Δθ.

Accordingly, to ensure that the desired value of Δθ is achieved, it is important to control the sound stimulation on the basis of the relation θs=θL+Δθ so that the sound stimulation is generated at the appropriate timing θs.

In other words, the instant of the zero-crossing as the fundamental frequency component 190 of the waveform pattern of the transmittance 15 repeatedly generated by the LCD element 60 increases is set as the timing B representative of the phase of the light stimulation 200, and the drive signal 45 is supplied to the sound-emitting device 160 at the timing C shifted from the timing B by the phase difference Δθ by a audio-visual control device.

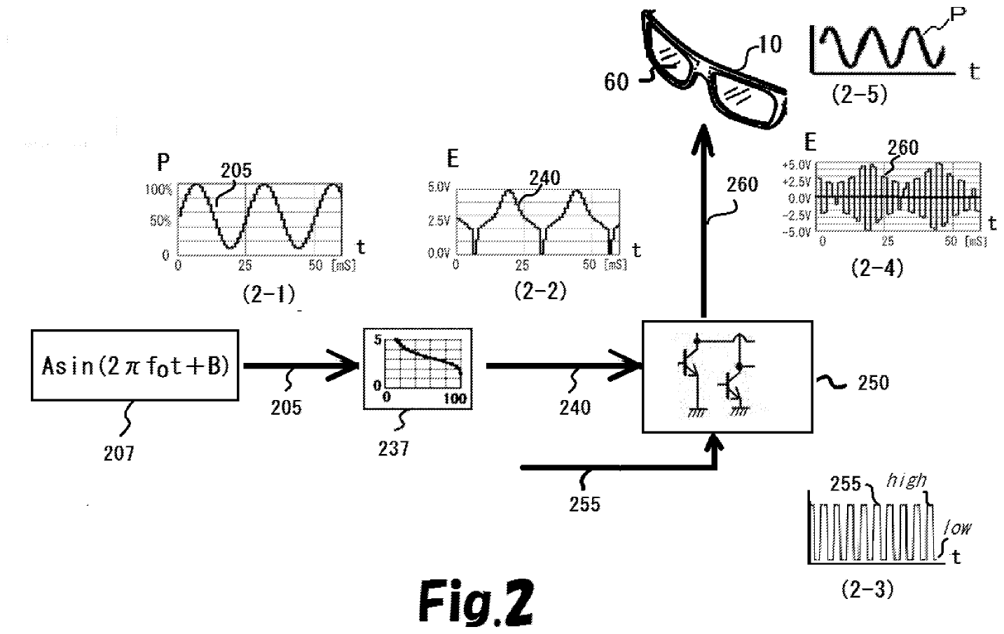

FIG. 2 is a diagram of a feed-forward control system that generates “continuous light containing one or multiple fundamental frequencies fn and harmonic frequencies fm of the fundamental frequencies fn” in the LCD sunglasses of the present invention.

First, a brief overview of the control system will be given.

The feed-forward control system has an inverse mapping correction part 237 as a nonlinear compensation element 265, and controls sampling on a fixed sampling interval. The object of control is the LCD element 60 with nonlinear characteristics that is mounted to the LCD sunglasses 10. For the transmittance target value 205 as the control target, the specific transmittance P [%] expressed by normalizing the transmittance is used. An operation amount is the voltage E as an absolute value 240 of the voltage applied to the LCD element 60. In the case of adopting a DC-driven LCD element 60, the voltage E is applied to the LCD element 60.

However, at the time of filing the present invention, an AC-driven LCD element 60 is typical. Accordingly, FIG. 2 comprises a polarity reversal part 250 that reverses the polarity of the voltage E of the absolute value 240 of applied voltage on the basis of the timing of a polarity reversal signal 255, which is a square wave of 200 Hz or higher. As a result, an AC voltage 260 of +E is applied to the LCD element 60. Note that in FIG. 2, the time series graphs (2-2) and (2-4) are plotted with the maximum value of E set to 5 [V].

In the related art, the “actual transmittance Q”, that is, “the percentage of the value obtained by dividing the illuminance Lo of the transmitted light by the illuminance Li of the incident light” is often used to indicate transmittance.

However, in the present invention, the waveform pattern of the transmittance from moment to moment requires a normalized representation. Accordingly, in the present invention, the “specific transmittance P” is used to indicate transmittance, being defined as “the percentage of the value obtained by dividing the illuminance Lo(t) of the transmitted light from moment to moment by the illuminance Lo_max of the transmitted light at maximum transmittance” under the condition of constant incident light Li. In other words, in the present invention, the “state of maximally controlled transmittance” is normalized as the specific transmittance P=100%, regardless of the model of the LCD element 60 employed.

Hereinafter, the functions of the control system will be described sequentially, following the control block diagram in FIG. 2.

In the transmittance target generator 207 that is first in the block configuration, the transmittance target value 205 at each time of sampling is generated on the basis of a “time function F(t) on a repeating cycle T0 [ms]” or “digital data pre-registered in an array or other numerical table”. For the transmittance target value 205, the specific transmittance P is used, with the range set from 0% to 100%. The time series graph (2-1) drawn in FIG. 2 is an example of the waveform pattern of the transmittance target value 205.

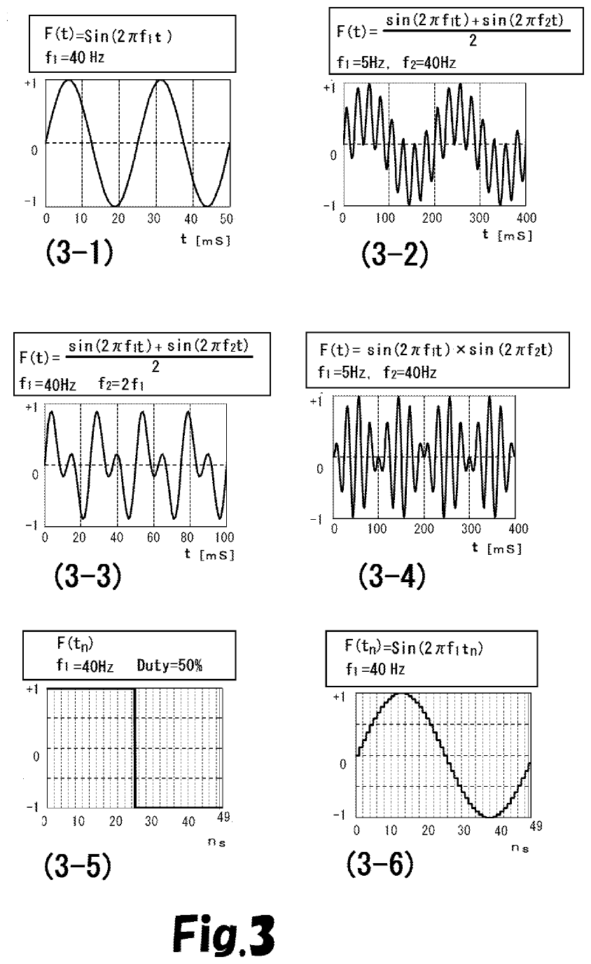

As demonstrated in FIG. 2 and the example of the fundamental waveforms WF in FIG. 3 described later, the transmittance target value 205 of the present invention needs to be a variable (such as an integer or floating point, for example) with a structure that can represent not only binary values but also multiple values beyond binary values. This is significantly different from the configuration of the LCD sunglasses of the related art in which only the two values of “high” and “low” are switched in only two steps. This is one feature of the LCD sunglasses of the present invention.

Specifically, the audio-visual control device 50 (see FIG. 6) uses a variable with a data structure expressing two or more numerical value levels as the transmittance target value 205 of the LCD element 60 and/or the absolute value 240 of applied voltage.

The inverse mapping correction part 237 that is second in the block configuration functions as a nonlinear compensation element 265 that compensates for the nonlinear characteristics of the LCD element 60. The inverse mapping correction part 237 functions to calculate the voltage E to be applied to the LCD element 60 to achieve the desired specific transmittance P of the transmittance target value 205. When actually incorporated into the control system, a single approximation formula or approximation formulas divided into multiple ranges of input values are applied to represent a monotonically increasing or monotonically decreasing nonlinear inverse mapping relationship.

In the present invention, the input of the inverse mapping correction part 237 is the transmittance target value 205 taking a value in the range from 0% to 100%, and the output is the voltage E as the absolute value 240 of the voltage applied to the LCD element 60. In one example, the range of the voltage E takes a value from 0 V to 5 V. Note that the range of the voltage E is specified by manufacturer recommended values in the component specifications of the LCD element 60 employed. If an excessively high voltage is applied, the LCD element 60 may be damaged instantaneously.

The time series graph (2-2) in FIG. 2 is an example of the waveform pattern of the absolute value 240 of applied voltage. In this way, the nonlinearity of the LCD element 60 requires the application, to the LCD element 60, of a voltage in a waveform pattern that is not similar to the transmittance target value 205.

The polarity reversal part 250 that is third in the block configuration functions to convert the voltage E [V] as the absolute value 240 of the voltage (namely, a DC voltage) applied to the LCD element 60 into the AC voltage 260 of ±E [V] by using a polarity reversal signal 255 of frequency F2.

The time series graph (2-3) in FIG. 2 is an example of the polarity reversal signal 255 as a square wave that switches between the two values “high” and “low”, and the time series graph (2-4) is an example of the waveform pattern of the AC voltage 260. A frequency of 200 Hz or higher is recommended as the frequency F2 of the square wave adopted as the polarity reversal signal 255. The reason is that, according to the specifications of the LCD sunglasses of the present invention, light stimulation is generated in the range of fundamental frequencies from F1=1 Hz at minimum to F1=100 Hz at maximum available for SSVEP. In other words, in that case, F1 and F2 produce a beat frequency F3=F1±F2. If the beat frequency F3 is 100 Hz or lower, brain waves may be generated as noise by entrainment derived from unintended beat frequencies. Therefore, F2 is set to 200 Hz or higher to keep noise from being generated in brain waves due to a beat frequency of 100 Hz or lower as F3.

As above, the audio-visual control device 50 calculates the absolute value 240 of applied voltage from the transmittance target value 205 of the transmittance 15 of the LCD element 60, and applies, to the LCD element 60, the AC voltage 260 with the polarity of the absolute value 240 of applied voltage reversed at the reversal frequency F2.

FIG. 3 illustrates various examples of fundamental waveforms WF usable for generating the transmittance target value 205 in the transmittance target generator 207. The following illustrates examples of time series graphs and formulas in which the fundamental frequency WF ranges from −1 to +1. In the example of the time function, design changes can be made to the parameters of the fundamental waveform WF by adjusting parameters such as the amplitude, frequency, and phase of the given trigonometric functions, by changing the coefficient multiplied by each term to a number other than 1, or by adding and superimposing one or more trigonometric functions. Furthermore, a time function other than a trigonometric function may be used insofar as the function repeats on the cycle T0 and ranges from −1 to +1.

In the example of using “discrete value data pre-registered in an array or other numerical table”, for example, the fundamental waveform WF with a repeating frequency of Δθ Hz on the cycle T0=25 [ms] is divided into 50 segments with a sampling interval Ts=0.5 [ms], for example. In this case, each of the 50 divided sampling intervals is assigned a sequential number, designated the loop number ns, from ns=0 to ns=49. The diagrams are merely examples, and design changes can be made to the repeating frequency (or cycle T0), sampling interval Ts, and number of divisions.

The transmittance target generator 207 is designed such that the parameters of the fundamental waveforms WF described above are adjusted first, and then the transmittance target value 205 is further adjusted to a final value range from 0% to 100% before being outputted from the transmittance target generator 207. As long as the numerical value of the transmittance target value 205 falls in the range from 0% to 100%, it does not matter if the numerical value is biased toward 0%, 100%, or any other value, or if the numerical value is discontinuous over time.

Even if the transmittance target value 205 is set to 0%, the transmittance control result of the LCD element 60 will not be completely opaque (0% transmittance), and the transmitted light Lo cannot be dark because some incident light Li will leak out.

The time series chart (3-1) in FIG. 3 illustrates the example of a single sine wave. Although the diagram illustrates an example formula of a sine wave with a frequency f1 of Δθ Hz, the frequency f1 may be changed in the range from 1 Hz to 100 Hz or the like, or a parameter for adjusting the phase of the sine wave can be added. Specifically, the audio-visual control device 50 describes the control target of the transmittance 15 of the LCD element 60 as a time function F(t) of time t containing one fundamental frequency fn.

The time series chart (3-2) in FIG. 3 is an example of superimposing sine waves with two fundamental frequencies fn. The numerical values [Hz] of the fundamental frequencies fn may be changed. The number of fundamental frequencies fn can be increased to three or more, and control parameters for amplitude and phase can be added to the sine wave of each fundamental frequency fn.

The time series chart (3-3) in FIG. 3 is an example of adding and superimposing the second harmonic at double the frequency onto a single fundamental frequency fn. Multiple m-th harmonics of the fundamental frequency may also be added and superimposed. Furthermore, one or more m-th harmonics of each of two or more fundamental frequencies may be superimposed similarly. The numerical values [Hz] of the fundamental frequencies fn may be changed. Parameters for adjusting amplitude and phase can also be added to the sine wave of each fundamental frequency fn and m-th harmonic.

The time series chart (3-4) in FIG. 3 is an example of phase-amplitude coupling expressed as the product of sine waves with two fundamental frequencies fn. The numerical values of the fundamental frequencies fn may be changed. Parameters for adjusting amplitude and phase can also be added to the sine wave of each fundamental frequency fn.

The coefficient multiplied by each term may also be changed to a number other than 1 in the above.

Specifically, in these three examples, the audio-visual control device 50 describes the control target of the transmittance 15 of the LCD element 60 as a time function F(t) containing a plurality of fundamental frequencies fn.

The time series chart (3-5) in FIG. 3 is the waveform pattern of a square wave expressed with “discrete data”. The numerical values of the waveforms corresponding to the loop number ns for each sampling time tn in the cycle T0 are defined in a data format that can be implemented in a computer, such as array variables, for example. With this format, the upper and lower limits, duty cycle, and phase can be defined freely for a square wave. In addition to square waves, staircase waveforms and saw waves as well as waveforms calculated from random numbers can be included to freely define a repeating waveform of cycle T0.

The time series chart (3-6) in FIG. 3 is an example of defining a single sine wave as another example of expressing a waveform pattern with “discrete data”. It is possible to not only modify waveform patterns based on the amplitude and phase of the fundamental frequencies of two or more sine waves and any number of harmonics, but also define waveform patterns with any waveform, such as a staircase waveform, superimposed onto the sine wave, for example.

In other words, in the above two time series chart examples, a periodic waveform pattern that repeats on the cycle T0, in other words, a numerical value for each sampling time tn for the loop number ns from the minimum value 0 to the maximum value, can be defined. Specifically, the audio-visual control device 50 describes the control target of the transmittance 15 of the LCD element 60 as a periodic waveform pattern F(tn) with a defined value at each sampling time tn.

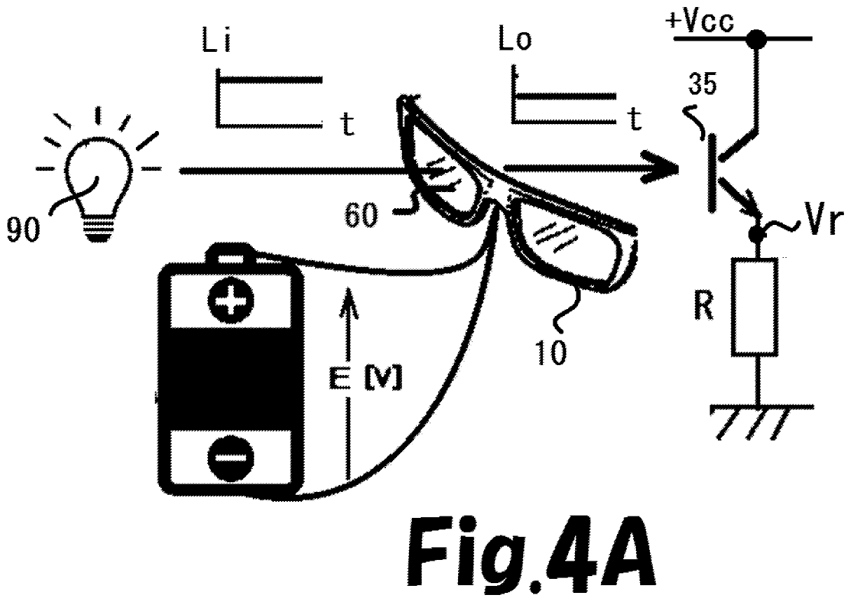

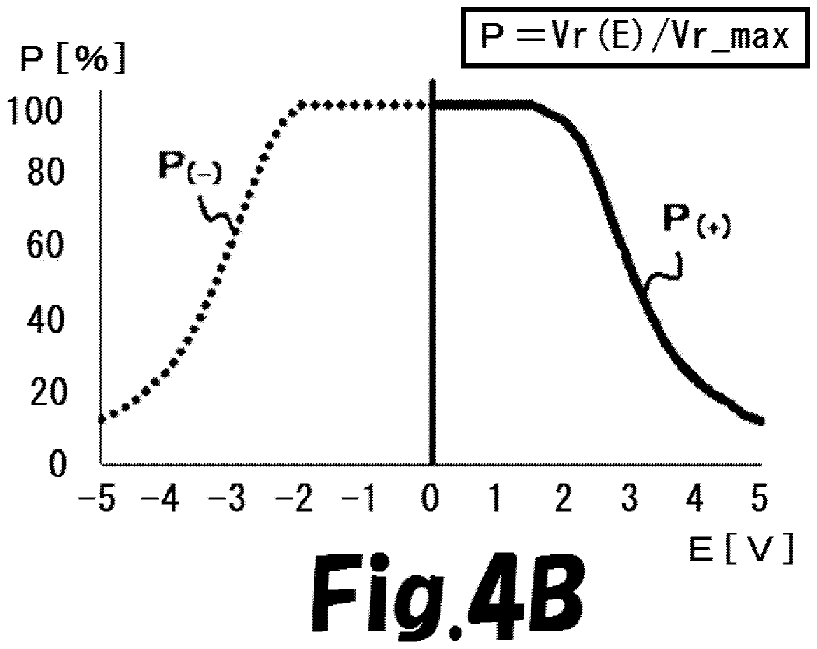

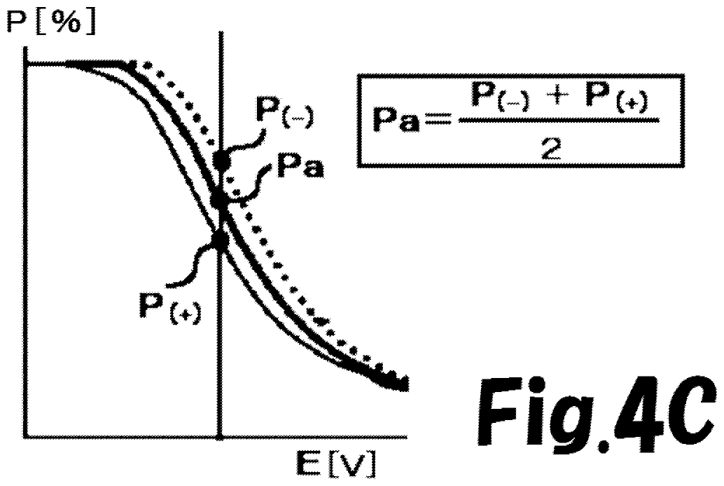

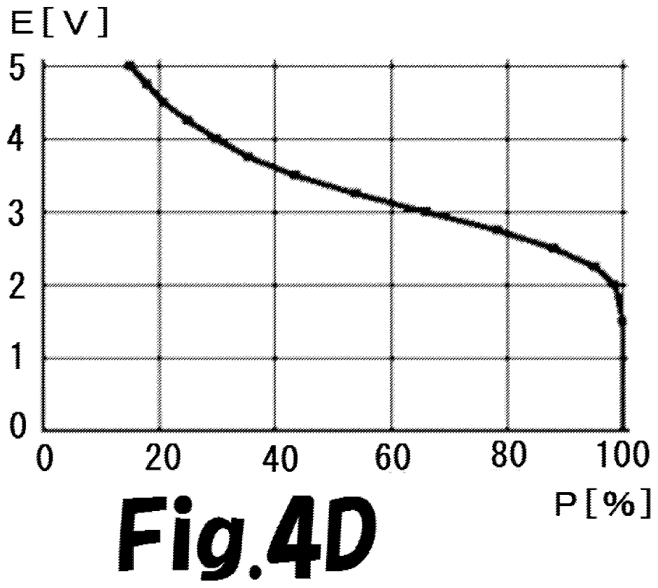

FIG. 4A schematically illustrates a configuration of a device for measuring the mapping characteristics from the applied voltage E to the specific transmittance P of the LCD element 60 built into the LCD sunglasses 10, which is necessary to design the inverse mapping correction part 237.

The illuminance Li of incident light entering the LCD element 60 from the light source 90 of constant brightness is constant, and the illuminance of the transmitted light Lo is also constant when a constant DC voltage E is applied to the LCD element 60. A voltage drop Vr in the emitter resistance of the semiconductor illuminance sensor 35 (model number NJL7502L, for example) is measured by using the illuminance of the transmitted light Lo. The value of the voltage Vr is proportional to the illuminance of the transmitted light Lo incident on the semiconductor illuminance sensor 35.

In the present invention, the normalized specific transmittance P is used for the transmittance. Accordingly, the applied voltage E is adjusted to set the voltage Vr of the semiconductor illuminance sensor 35 to a maximum voltage Vr_max when the transmitted light Lo is at maximum brightness.

Let a measured value Vr(E) be the voltage Vr of the semiconductor illuminance sensor 35 for an applied voltage E [V].

In this case, the specific transmittance P for the applied voltage E of the LCD element to be measured is calculated by P=Vr(E)/Vr_max.

FIG. 4B illustrates one example of measurement of the applied voltage E in a range free of damage by overvoltage. Specifically, the applied voltage E is measured from −5 [V] to +5 [V] every 0.5 V, and the curve smoothly connects the measured values of specific transmittance P for each applied voltage E.

FIG. 4C illustrates the graph in FIG. 4B replotted with the absolute value of the applied voltage E on the horizontal axis. As illustrated in the drawing, in some cases, the shape of the graph may be inconsistent between the specific transmittance P (+) when the applied voltage E is positive and the specific transmittance P (−) when the applied voltage E is negative. FIG. 4C illustrates the three graphs of the specific transmittance P (+), the specific transmittance P(−), and the average Pa of the two.

Hereinafter, the graph of the curve of the average Pa is designated the mapping graph 230. In the present invention, the LCD element 60 is used only in the segment where the mapping graph 230 has monotonically increasing or monotonically decreasing characteristics.

FIG. 4D illustrates the mapping graph 230 with the X and Y axes reversed, which is designated the inverse mapping graph 235. Since the mapping graph 230 has monotonically increasing or monotonically decreasing characteristics, the inverse mapping graph 235 also has monotonically increasing or monotonically decreasing characteristics.

The inverse mapping graph 235 indicates the applied voltage E required to achieve a desired specific transmittance P [%].

Consequently, If the transmittance target value 205 ranging from 0% to 100% generated by the transmittance target generator 207 is specified on the X axis of the inverse mapping graph 235, the absolute value 240 of applied voltage E to be given can be read off the Y axis.

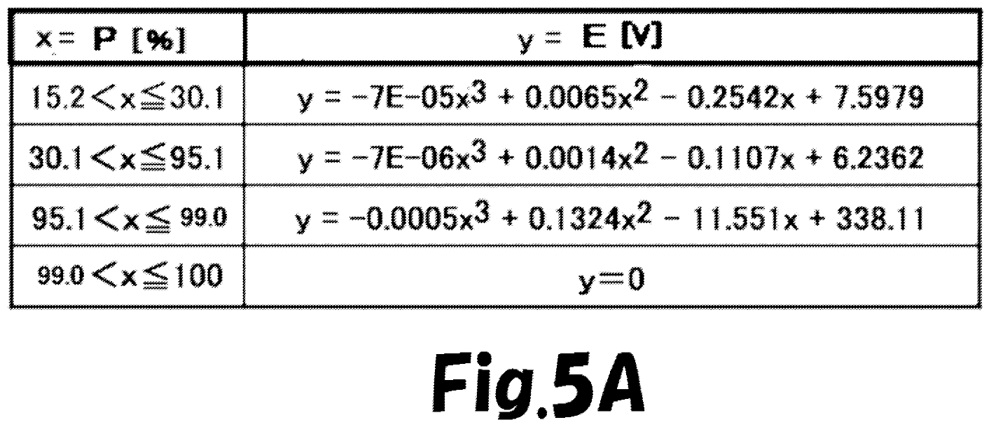

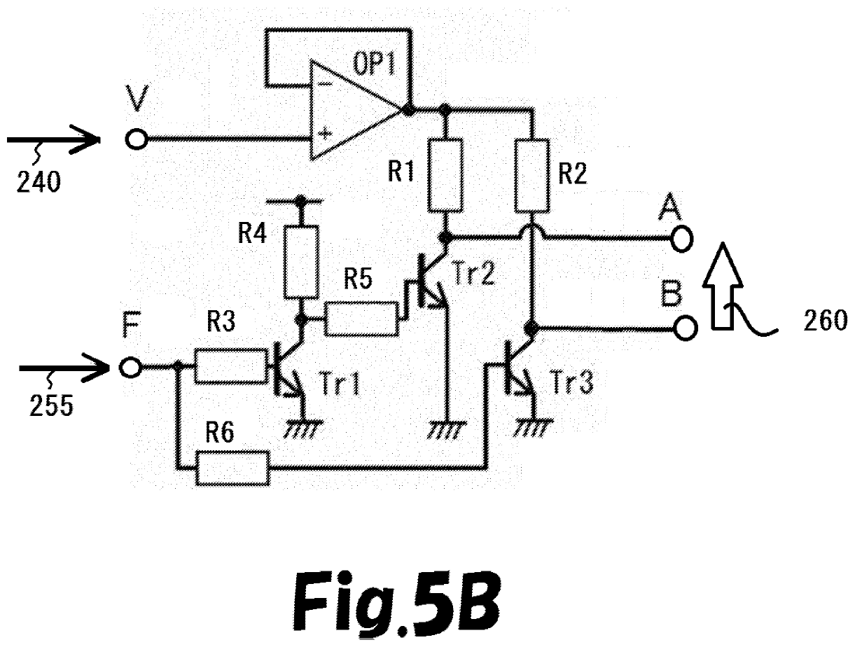

FIG. 5A is an example of how the inverse mapping graph 235 can be implemented in a computer for control by dividing the domain on the X axis of the inverse mapping graph 235 into multiple segments, each of which described by an approximation formula that calculates the value of the range on the Y axis in each segment.

If these approximation formulas are implemented in a microcomputer for control that performs the calculations of the inverse mapping correction part 237, the absolute value 240 of applied voltage can be computed as the output from the transmittance target value 205 as the input.

FIG. 5B illustrates an example of using readily available electronic components to configure the polarity reversal part 250. The absolute value 240 of applied voltage is inputted into a terminal V. The LCD element 60 driven at low speeds is a high-impedance component and thus can be driven by the output voltage of an operational amplifier OP1 as a voltage follower.

After connecting each electrode that drives the LCD element 60 to terminals A and B, a square wave that switches between logical values “high” and “low” at 200 Hz, for example, is applied to a terminal F as the polarity reversal signal 255. This causes transistors Tr2 and Tr3 to turn on and off alternately, and thus the two electrodes of the LCD element 60 are alternately subjected to a transistor residual voltage close to the absolute value 240 of applied voltage and a ground potential of 0V. As a result, the AC voltage 260 with a half amplitude that is substantially the absolute value 240 of applied voltage is applied between the two electrodes of the LCD element 60.

Although transistors are used for Tr2 and Tr3 in the example in FIG. 5B, the design changes may also be made to a FET or other element to reduce the residual voltage during the period when the transistor element is ON (conducting).

Based on the disclosure above, the circuit design can be changed or subjected to design changes, such as conversion to an LSI chip.

The above describes a method of entrainment by light stimulation 200 using the LCD sunglasses 10.

Hereinafter, a method of lessening variations in therapeutic effects in the sensory stimulation therapy device 20 of the present invention is described.

In the related art, it is known that when separate stimuli are phase-synchronized and applied to two separate locations in the brain, it is necessary to note the phase difference between the two stimuli. For example, as already described, the results of a test applying phase-synchronized stimulation to two locations at the front and back of the head were published in 2019. Specifically, when stimuli of opposite phase (phase difference ±180 degrees) are applied to the two locations, a temporary cognitive impairment occurs in healthy subjects. This may be considered a serious adverse event.

On the other hand, as the inventor of this invention experienced by chance as a subject in the process of conducting data measurement work, when the phase difference Δθ between light and sound stimulation exceeds the range from −90 to +90 degrees, unpleasant sensations such as headache and intense sleepiness may occur. This may not rise to the level of a “serious” adverse event. However, if there is a risk of “unpleasant” adverse events even when deviating from the phase difference range of Δθ=+90 degrees which is half the phase difference of ±180 degrees indicated in the above research case study, then this is a quality control issue that should be noted and guarded against.

Accordingly, the following focuses on the existence of the light stimulation onset delay θL due to poor response of the LCD element to ensure the reproducibility of Δθ as a condition for entrainment in each treatment. In other words, “developing a therapeutic device that achieves the desired Δθ” according to the present invention is a solution for lessening variations in therapeutic effects.

Furthermore, according to the present invention, the desired value of Δθ achieved as the phase difference Δθ between light and sound stimulation can be managed to not deviate from the range of −90 to +90 degrees. In other words, the absolute value of the phase difference Δθ is managed to be less than ¼ of one cycle of the light stimulation 200.

FIG. 6 is a configuration example of an apparatus used for system development and quality control of the sensory stimulation therapy device 20. The therapeutic device for which a system is to be developed is briefly summarized below using FIG. 6.

A sensory stimulation therapy device 20 for preventing and treating neurological disorders involving impairment of functional connectivity and provided with a structure enabling a subject 1 of treatment to visually perceive a surrounding environment during treatment action, wherein the sensory stimulation therapy device 20 comprises a visual stimulator 30, an auditory stimulator 40, and an audio-visual control device 50, the visual stimulator 30 is LCD sunglasses 10 that generate light stimulation 200 by controlling the transmittance 15 of light incident from the surrounding environment transmitted through an LCD element 60 by placing the LCD element 60 as a lens 80 of 2 eyes or wide 1 eye LCD sunglasses, the auditory stimulator Δθ is a sound-emitting device 160 such as earphones 168 that generate sound stimulation 210 on an audible frequency, and the audio-visual control device 50 is control means that generates the light stimulation 200 by supplying an applied voltage of an AC voltage 260 (or a DC voltage, in a case of a DC-driven LCD element) to the LCD element 60, generates the sound stimulation 210 by supplying a drive signal 45 driven by an audio amplifier to the sound-emitting device 160, and controls the phase difference Δθ from the light stimulation 200 to the sound stimulation 210.

In FIG. 6, the portions of the visual stimulator 30, the auditory stimulator 40, and the audio-visual control device 50 are the sensory stimulation therapy device 20. The remaining portions are equipment used for system development and quality control of the sensory stimulation therapy device 20. A PC 430 is used to develop a program of a microcomputer 170 for control included in the audio-visual control device 50 and to analyze the digital data of signals collected by an oscilloscope 500 to measure and inspect characteristics. A smartphone 302 is a remote control terminal for using a built-in application 355 to change control parameters of the microcomputer 170 for control included in the audio-visual control device 50.

Hereinafter, the visual stimulator 30, the auditory stimulator 40, the audio-visual control device 50, the PC 430, the oscilloscope 500, and the smartphone 302 in the present example is each described in detail.

First, the visual stimulator 30 is the LCD sunglasses 10 that generates the light stimulation 200 by controlling the transmittance 15 of light incident from the surrounding environment transmitted through the LCD element 60 by placing the LCD element 60 as the lens 80 of the 2 eyes or wide 1 eye LCD sunglasses.

For the LCD element 60, the transmittance of the entire transmissive surface should change as uniformly as possible without bias when the applied voltage is changed. An LCD element 60 that causes transmittance changes concentrated on only a portion of the transmissive surface may cause problems with entrainment in brain waves, and thus should be eliminated as defective. Furthermore, the monotonically increasing or monotonically decreasing nonlinear curves represented by the inverse mapping graph 235 must be reproducible.

For example, active glasses for watching 3D movies with specifications compatible with 3D display devices with frequencies of 144 Hz or higher are available for sale on the Internet for about $20. Even the LCD element 60 incorporated in such an inexpensive commercial product, if inspected at the receiving stage and found to meet the above adoption requirements, can be used for entrainment by continuous waves with frequencies from 1 Hz to 100 Hz in the present invention.

LCD sunglasses for dynamic field training with a wide field of view are commercially available for about $400 to $800, and a high-performance LCD element 60 for optical experiments, costing more than $1000 for one eye alone, is also available. If the above adoption conditions are met, it may be possible to make sunglasses that can be set to a higher brightness level and to improve the accuracy of transmittance control. Even if the LCD element 60 itself is purchased as a component, a suitability determination is still mandatory during acceptance inspection at the receiving stage.

Second, the auditory stimulator Δθ is used to apply the same sound stimulation 210 to the left and right ears. Entrainment is possible even with inexpensive stereo earphones 168 commercially available for a few dollars. However, to efficiently use sound stimulation 210 containing frequency components spread over the audible range approximately from Δθ Hz to 20 kHz, a sound-emitting device 160 placed close to the ears as a high-fidelity audio source with as excellent a frequency response and as wide a dynamic range as possible is preferable. Besides wired earphones 168 or headphones, small speakers may also be driven near the ears.

Conversely, if acoustic data is transmitted wirelessly to earphones, headphones, or speakers, a non-negligible phase delay may occur between the output of the sound stimulation signal from the control device and the actual hearing of the sound, and thus is not recommended.

Also, placing speakers at a distance far from the subject of treatment, gathering a large number of subjects viewing a common light stimulation 200 in a large room to listen to sound stimulation 210 from speakers, or exposing subjects of treatment and caregivers to light stimulation 200 and sound stimulation 210 under conditions in which the distance from the speakers varies, such as while dancing, is not recommended. This is because in these use cases, Δθ may exceed 90 degrees due to the phase delay caused by sound propagating through the air from the sound-emitting device 160 to the location of the subject.

Third, the audio-visual control device 50 is an example of a component that is easily obtainable by anyone. Common components that anyone can purchase at an electronic parts store on the Internet are used for the microcomputer 170 for control and the electronic circuit components. All parts were available online at Akizuki Denshi Tsusho Co., Ltd., in Akihabara, Japan. This versatile disclosure example enables a person skilled in the art to develop a system by making design changes to use components and development equipment that are readily available and easy to use.

For the microcomputer 170 for control, the commercial development kit AE-ESP-WROOM-02-DEV incorporating ESP8266EX with a Wi-Fi communication function is used. A small Wi-Fi antenna is also built in, enabling communication over Wi-Fi with the external smartphone 302. A UART for serial communication is also built in, enabling program development from the external PC 430. Input/output (I/O) is also built in, enabling I2C communication in addition to digital output (DO1, DO2, DO3, and so on). An analog output AO1 is outputted from a D/A converter MCP4725 via FXMA2102, a commercially available element that changes the signal voltage level for I2C communication, and the absolute value 240 of applied voltage is directed through the polarity reversal part 250 detailed in FIG. 5B. The polarity reversal signal 255 of the polarity reversal part 250 is outputted from the digital output DO1. The digital output DO2 outputs the phase synchronization signal S, and the digital output DO3 outputs the sound stimulation 210.

Fourth, the PC 430 is a commercially available desktop PC with a general-purpose OS installed.

To develop a control program of the audio-visual control device 50, the known development system Arduino (R) is installed on the PC 430 and used. To collect characteristics data of the LCD element 60 and measure the parameter θL, the numerical analysis software MATLAB (R) is installed.

Fifth, to measure characteristics data of the LCD element 60, a commercially available oscilloscope 500 with a data logging function is used to collect data. The measured digital data is outputted as text data to a USB memory and transferred to MATLAB (R) on the PC 430 for analysis. In the present example, Iwatsu DS-5105B is used as the oscilloscope 500.

Note that the oscilloscope 500 is used to measure two signals: the phase synchronization signal S of the digital output DO2 of the audio-visual control device 50 and the emitter voltage drop Vr of the semiconductor illuminance sensor 35.

The time series graph (6-1) in FIG. 6 illustrates an example of data collection of the phase synchronization signal S and the waveform pattern of the emitter voltage drop Vr of the semiconductor illuminance sensor 35 detected as the light stimulation 200.

The time series graph (6-2) in FIG. 6 illustrates an example of plotting the phase synchronization signal S in comparison with the waveform pattern of the fundamental frequency component 190 of Vr calculated using the FFT and the inverse FFT in MATLAB (R). In the times series graph, point B is plotted with delayed phase relative to point A, and this phase difference corresponds to the phase delay OL from points A to B.

Note that the method of calculating the waveform pattern of the fundamental frequency component 190 of the voltage Vr using the FFT and the inverse FFT in MATLAB (R) and the method of plotting the phase synchronization signal S in comparison with the timings are already described in FIG. 1A as the procedure for measuring the phase delay OL from point A to B.

The apparatus in FIG. 6 can also be used for the measurement of the nonlinear characteristics of the LCD element 60 already described in FIG. 4A.

Specifically, with the polarity reversal signal 255 of frequency F2=200 Hz, for example, applied to the polarity reversal part 250 in FIG. 6, a test level of the applied voltage E [V] is repeatedly applied from 0 V to 5 V, increasing at 0.5 V intervals, for example, as the absolute value 240 of applied voltage from the computer for control.

In this way, the numerical value of the emitter voltage drop Vr of the semiconductor illuminance sensor 35 can be read efficiently as a digital value using the cursor of the oscilloscope 500. Using this method, calculating the specific transmittance P (+) when a positive voltage (+E) is applied, the specific transmittance P (−) when a negative voltage (−E) is applied, and the average specific transmittance Pa and collecting the measurement data in FIGS. 4B and 4C are easy.

In FIGS. 4A to 4D, the basics of nonlinear compensation of the LCD sunglasses 10 are introduced. In this context, in the conceptual explanation of the method of measuring the nonlinear characteristics of the LCD element 60 referencing FIG. 4A, the example of applying a DC voltage as the test level of the applied voltage E [V] is used for simplicity. However, from the standpoint of product development, the shape of the mapping graph 230 in FIG. 4C illustrating the nonlinear characteristics of the LCD element 60 changes slightly for each product due to the influence of the frequency F2 of the polarity reversal signal 255 and the circuit constant of the polarity reversal part 250 in addition to the characteristics of the LCD element 60 itself.

Thus, for quality assurance at the actual product development and manufacturing stages, it is desirable to perform measurement and inspection using the electronic circuit of the polarity reversal part 250 used in the actual product measure while applying the test level of the applied voltage E [V].

If inspection is performed at the initial manufacturing stage of the LCD sunglasses 10, and the shape of the mapping graph 230 deviates significantly from a reference value established at the design stage, the LCD element 60 is eliminated from the production line as a defective product suspected of developing a product defect during manufacturing.

However, if the shape of the mapping graph 230 is monotonically increasing or monotonically decreasing and the deviation from the reference value is minor, a simple variation in the manufacturing of the LCD element 60 is likely. If so, shipment as a superior good is possible in many cases by adjusting the approximation formulas in FIG. 5A to conform to the shape of the measured mapping graph 230. In any event, by performing inspection to check quality, the risk of shipping an inferior good can be reduced.

Sixth, the smartphone 302 can be used as a remote control terminal for using a built-in application 355 to change the control parameters of the microcomputer 170 for control included in the audio-visual control device 50.

In the present example, the waveform pattern of the transmittance target value 205 as a parameter for control and the desired set value of the phase difference Δθ for phase synchronization between light and sound can be written into a control program of the audio-visual control device 50 as predefined numerical values, and thus the function of remote control by the smartphone 302 is not essential to the present invention.

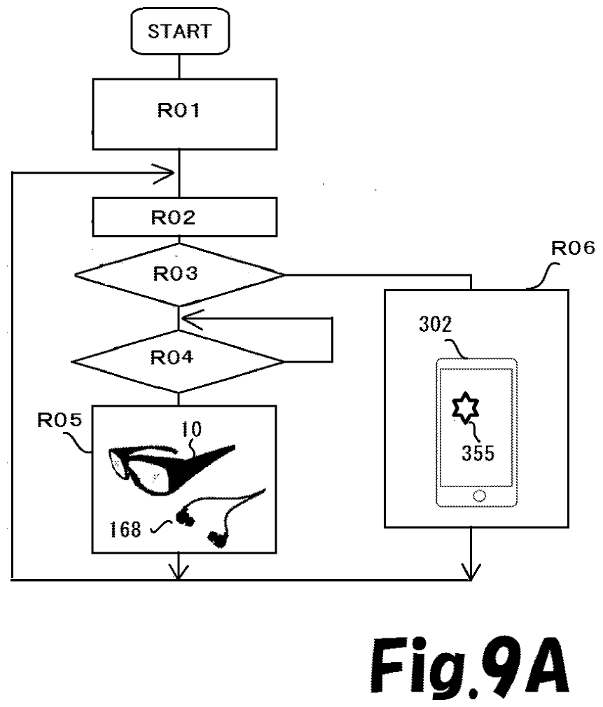

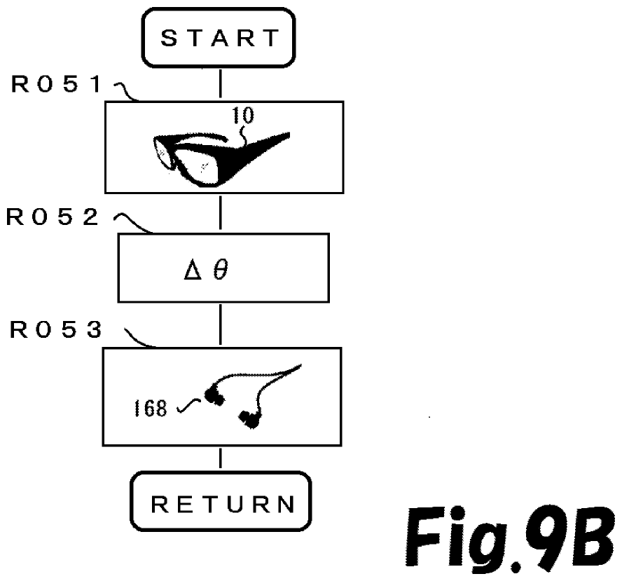

However, to facilitate understanding of the versatility of the present invention, an example of incorporating software for rewriting control parameters remotely from the smartphone 302 is described later using FIG. 9A. Through remote control performed using the smartphone 302 in this way, it is possible to switch the waveform pattern of the transmittance target value 205 by selecting from among the various fundamental waveforms WF exemplified in FIG. 3, change the upper and lower limits of the selected waveform pattern, switch a single fundamental frequency from Δθ Hz to 10 Hz, for example, or change the desired set value of the phase difference Δθ of the phase synchronization between light and sound. Furthermore, remote operations such as switching the measured “phase delay θL from points A to B” to a corresponding value in correspondence with these waveform patterns can also be performed using the application 355 installed in the smartphone 302.

As explained in FIGS. 1 to 6 above, a control system can be developed to control the transmittance of LCD sunglasses for entrainment of light stimulation by SSVEP using continuous light.

First, as explained in FIGS. 2 and 3, waveform data of the transmittance target value 205 of the LCD element 60 is generated.

Next, as explained in FIGS. 2, 4A, 4B, 4C, 4D, and 5A, the waveform data of the absolute value 240 of the applied voltage of the LCD element is calculated by substitution into the approximation formula of the inverse mapping correction part 237 that calculates the correspondence relationship between the desired transmittance and the applied voltage.

Furthermore, as explained in FIGS. 2 and 5B, the AC voltage 260 is generated in the polarity reversal part 250 that reverses the polarity of the applied voltage to apply the voltage to the LCD element 60.

In this way, transmittance control of the LCD sunglasses can be achieved by performing sampling control on a fixed sampling interval on the basis of the block diagram illustrated in FIG. 2.

The following describes an example of measuring the “light stimulation onset delay θL” that occurs in the LCD sunglasses in correspondence with the characteristics of the LCD element and the light stimulation waveform data while controlling the transmittance of the LCD sunglasses using the control apparatus described above.

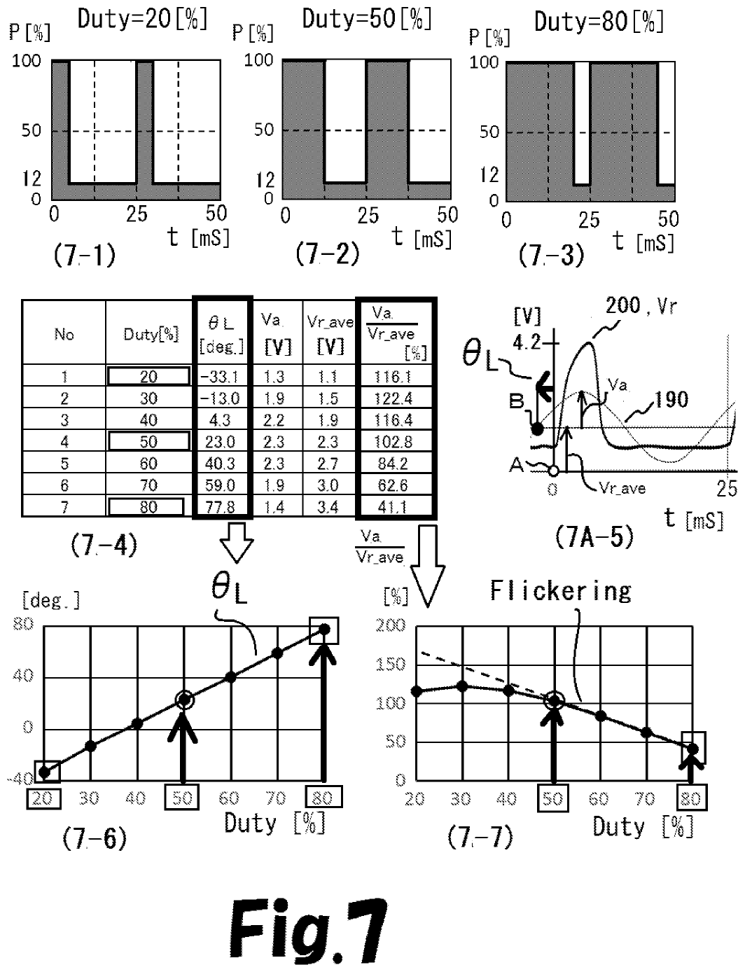

FIG. 7 illustrates waveform collapse when a square wave is used as the transmittance target value 205, and an example of measuring the light stimulation onset delay θL. The time series graph (7-1) is a square wave with a 20% duty cycle, the time series graph (7-2) is a square wave with a 50% duty cycle, and the time series graph (7-3) is a square wave with an 80% duty cycle. In all cases, the specific transmittance P ranges from 12% to 100% and the repeating frequency is Δθ Hz. In other words, with the characteristics of the LCD element 60 in this example, the control result does not go to 12% or below even if the transmittance target value 205 is lowered below P=12%. The transmitted light Lo cannot be dark because transmitted light at or above 12% always remains, and the light stimulation 200 is continuous light.

Such a square wave (which perhaps should be called a two-step staircase waveform) is outputted from the transmittance target generator 207 in FIG. 2 while varying the duty cycle from 20% to 80% in increments of 10% to measure the light stimulation onset delay θL.

The results of measuring θL analyzed in MATLAB (R) on the basis of the voltage Vr measured by the semiconductor illuminance sensor 35 are indicated in the table (7-4). The column labeled Va in the table is the half amplitude of the fundamental frequency component 190 obtained by analyzing Vr in MATLAB(R). Also, Vr_ave is the average of the brightness of the transmitted light, which is an indicator of eye pupil opening. Thus, Va/Vr_ave is the ratio of the half amplitude of the fundamental frequency component to the average brightness reaching the eyeball, and in effect is a “flickering index” of the LCD sunglasses as perceived by the subject.

The time series graph (7A-5) in FIG. 7 is an example for measuring at the 20% duty cycle, and is a copy traced by hand on the screen from the plots of Vr representing the waveform pattern of the brightness of the light stimulation 200, the average Vr_ave thereof, and the waveform pattern of the fundamental frequency component 190 of Vr analyzed in MATLAB (R), which were displayed superimposed on the screen. As shown in the graph, the light stimulation 200 transmitted through the LCD element 60 becomes continuous light in which the waveform of Vr, representing brightness, has collapsed, and a “sharp square-wave light pulse” cannot be generated.

The graph (7-6) illustrates the results of measuring OL for each duty cycle. The onset delay θL=23.0 degrees at 50% duty cycle and θL=77.8 degrees at 80% duty cycle, approaching 90 degrees. Conversely, at 20% duty cycle, OL goes to −33.1 and is advanced rather than delayed. In this way, the light stimulation onset delay θL fluctuates greatly under the influence of the characteristics of the LCD element and the waveform pattern.

The graph (7-7) is the “flickering index” of the LCD sunglasses for each duty cycle. Flickering is 102.8% (substantially 100%) at 50% duty cycle, decreasing to 41.1% at 80% duty cycle. Conversely, flickering increases if the duty cycle is lowered, but since the amplitude Va of the Δθ Hz fundamental frequency component also decreases, flickering reaches a maximum at around 30% duty cycle.

As above, even if a square wave is used as the transmittance target value 205, the light stimulation 200 is continuous light with a distorted waveform. Nevertheless, entrainment with light is possible because the action of SSVEP occurs due to the fundamental frequency 190.

It has been anticipated for some time that changing the duty cycle would change the “flickering index” of the LCD sunglasses, but the influence of θL has been unknown.

The current analysis reveals that the value of OL also varies from −33.1 degrees to 77.8 degrees with a swing of 110 degrees. In the relation of the timing θs=θL+Δθ for outputting sound stimulation, therapeutic effects are affected if Δθ exceeds the range from −90 degrees to +90 degrees, and also considering that unpleasant adverse events begin to occur, this swing of 110 degrees in θL is too large to be ignored.

Consequently, to achieve the desired Δθ, it is necessary to properly evaluate the influence of θL and determine the timing θs for outputting the sound stimulation from the relation θs=θL+Δθ. Thus, it is essential to measure and ascertain the value of θL in advance, as in FIG. 7, in accordance with the waveform pattern of the transmittance target value 205 that controls the LCD element 60.

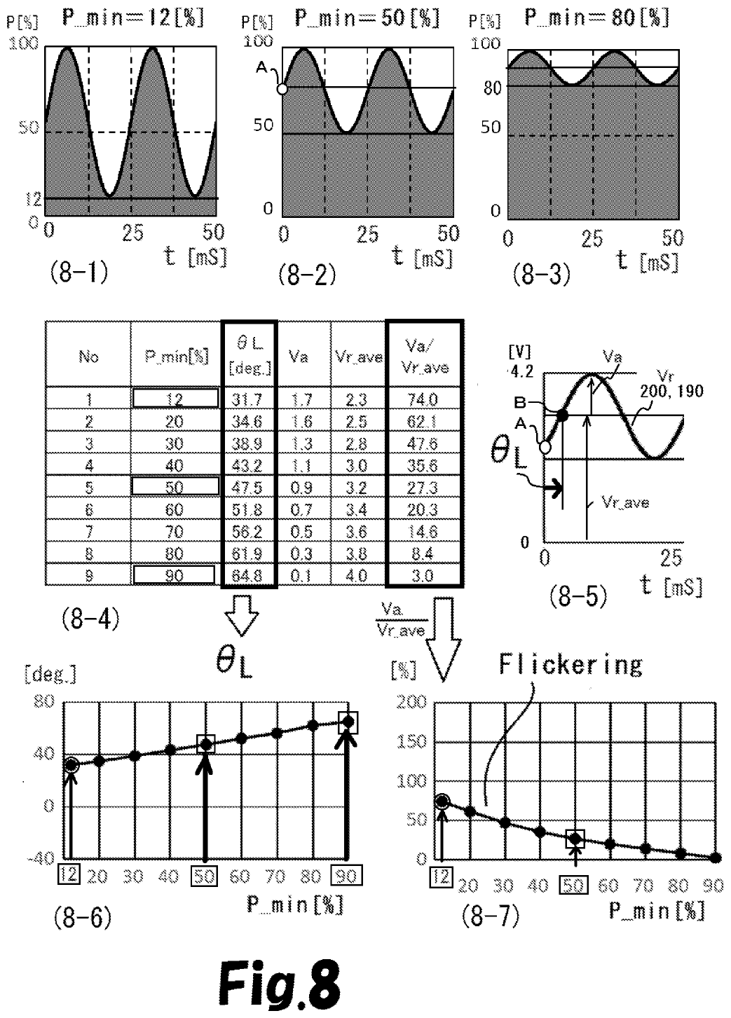

FIG. 8 illustrates an example of measuring the light stimulation onset delay θL when a sine wave is used as the transmittance target value 205. The time series graph (8-1) is a sine wave with an upper limit value of 100% and a lower limit value of 12% of the transmittance target value 205, the time series graph (8-2) is a sine wave with an upper limit of 100% and a lower limit of 50%, and the time series graph (8-3) is a sine wave with an upper limit of 100% and a lower limit of 80%. The repeating frequency is Δθ Hz. The expectation is that raising the lower limit value would raise the average transmittance of the LCD element 60 and thus increase the brightness of the sunglasses, whereas the amplitude would be smaller, and thus the “flickering index” of the LCD sunglasses would be smaller.

The light stimulation onset delay θL is measured by outputting the transmittance target value 205 from the transmittance target generator 207 in FIG. 2 with a waveform having such a sinusoidal change in brightness in which the upper limit value is fixed at 100% while the lower limit value is varied from 12% to 90% in increments of approximately 10%.

The results of measuring θL analyzed in MATLAB (R) on the basis of the voltage Vr measured by the semiconductor illuminance sensor 35 are indicated in the table (8-4). The meaning of each variable is the same as in FIG. 7.

The time series graph (8-5) in FIG. 8 is a copy of the plots for the case of measuring with an upper limit value of 100% and a lower limit value of 50%. The waveform pattern of the brightness of the light stimulation 200 indicated by Vr is slightly distorted, but is basically a sine wave. Analysis of the frequency spectrum of this Vr waveform shows that the amplitude of the 80 Hz harmonic component is approximately 15% of the amplitude of the Δθ Hz fundamental frequency component. Note that the fundamental frequency component 190 is a precise sine wave.

The two curves of the substantially sine wave Vr and the fundamental frequency component 190 substantially overlap, but are displayed in different colors on the PC screen and thus are distinguishable. However, since the time series graph (8-5) is a black and white copy, the two substantially overlapping curves are represented and drawn by a single, thick sine wave to make the drawing easier to read.

Note that, compared to the waveform pattern of the transmittance target value 205 in the time series graph (8-2), the phase of the sine wave in the time series graph (8-5) is delayed by θL. This mainly due not only to the slow response of the LCD element 60 but also to the asymmetric and nonlinear response in the rise and fall of the LCD element 60.

The graph (8-6) illustrates the results of measuring OL for each lower limit value of the specific transmittance P. Here, θL is 31.7 degrees for the lower limit value of 12%, but θL is 47.5 degrees for the lower limit value of 50%. Furthermore, if the lower limit value is raised to 90%, θL increases to 64.8 degrees. When only the lower limit value of the sine wave is varied, θL varies by 33.1 degrees. This variation is non-negligible, but demonstrates a lesser impact compared to the case of the square wave in FIG. 7.

The graph (8-7) illustrates the “flickering index” of the LCD sunglasses for each lower limit value of the specific transmittance P.

As originally expected, raising the lower limit value raises the average transmittance of the LCD element 60 and thus increases the brightness of the sunglasses, whereas the “flickering index” of the LCD sunglasses is smaller. However, note that the numerical values of the “flickering index” are lower overall compared to the graph (7-7) in FIG. 7.

In other words, when the lower limit value is adjusted using the sine wave in FIG. 8 as the transmittance target value 205, the numerical value of the “flickering index” tends to be lower than when the duty cycle is adjusted using the square wave in FIG. 7. This indicates a tendency of keeping the stimulation low to the eyes of the elderly whose vision function has declined.

Note that even if the lower limit value of the square wave is adjusted as the transmittance target value 205, the “flickering index”, θL, and the brightness of the LCD sunglasses can be adjusted. This means that the “flickering index” of the LCD sunglasses, θL, and the brightness of the LCD sunglasses can be adjusted by adjusting parameters such as the amplitude and average (or upper and lower limit values) of the transmittance target value 205 generated by the transmittance target generator 207 in FIG. 2.

Therefore, it would be appropriate for a medical professional to make the choice of whether to use a sine wave or a square wave as the fundamental waveform for creating eye-friendly light stimulation for the elderly after conducting clinical trials and collecting therapeutic effects and opinions from many subjects.

In this way, criteria for use as digital medicine need to be established through clinical trials, so that the control parameters of the LCD sunglasses are set to fundamental frequencies and waveforms suitable for each neurological disease to be treated.

Furthermore, if an elderly person as the subject feels that the LCD sunglasses flicker too much due to deteriorating eyesight, the “flickering” or brightness of the LCD sunglasses as represented by Vr_ave in FIGS. 7 and 8 can be adjusted. When using this therapeutic device, a treatment time suitable for the patient, such as 5 minutes, 30 minutes, or 1 hour, can also be adjusted. In other words, the light stimulation 200 to be applied for treatment can be adjusted at the discretion of the physician, enabling the physician to decide and prescribe the dosage of the digital medicine.

Hereinafter, an overall control routine of the sensory stimulation therapy device using LCD sunglasses is described. Specifically, θL measured as above is used to calculate θs by substituting the measured θL and the desired Δθ into the relation θs=θL+Δθ, and the timing for generating sound stimulation is controlled on the basis of θs.

FIG. 9A is a flowchart of overall control of a sensory stimulation therapy device using LCD sunglasses.

Routine R01 uses the Arduino(R) setup( ) function to initialize variables, input/output, and Wi-Fi.

Note that routines R02 to R06 loop the control process using the loop( ) function.

First, in routine R02, a process of reading a string from the Wi-Fi receive buffer is performed.

In the next routine R03, the receive buffer is searched for command characters giving an instruction to interrupt the light and sound entrainment control process, and if the control interrupt command characters are found, the flow proceeds to routine R06 and processes such as communicating with the application 355 on the smartphone 302 to change control parameters according to remote control instructions are performed, after which the flow returns to routine R02. If the control interrupt command characters are not found in routine R03, the flow proceeds to routine R04.

In the next routine R04, the sampling interval is managed to be 0.5 ms, for example. In Arduino (R), the elapsed time from the instant of starting program execution (hereinafter designated the “time”) can be read out in units of microseconds using the micros( ) function. Accordingly, the time of starting a new sampling interval is checked using the micros( ) function and logged. The micros( ) function is used to check whether 500 microseconds (i.e., 0.5 ms) have elapsed since the start time logged previously, and routine R04 is looped in standby until enough time elapses. If the standby continues and at least 500 microseconds have elapsed since the previous start time, the time is logged as the time of starting a new sampling interval.

Additionally, a loop number ns is incremented by 1 to indicate the ordinal number of the sampling interval among the number of divisions (such as 50 divisions, for example) obtained from dividing the light stimulation cycle TO into multiple sampling intervals, and the value of ns is reset to ns=0 if ns equals or exceeds the number of divisions.

If the loop number ns=0, the logical value “high” is outputted to the digital output terminal DO2 as the phase synchronization signal S. This is point A observed with the oscilloscope. If the loop number is not ns=0, the logical value “low” is outputted as the phase synchronization signal S.

In routine R05, the LCD sunglasses 10 and the earphones 168 are controlled. Specifically, as illustrated in FIG. 9B, the LCD sunglasses 10 are controlled in routine R051, calculations related to the phase difference Δθ are performed in routine R052, and the earphones 168 are controlled in routine R053. Details are described below.

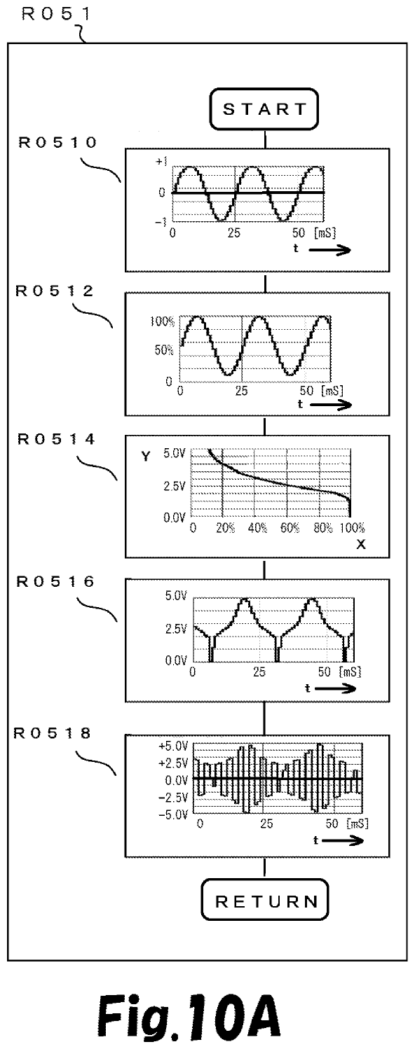

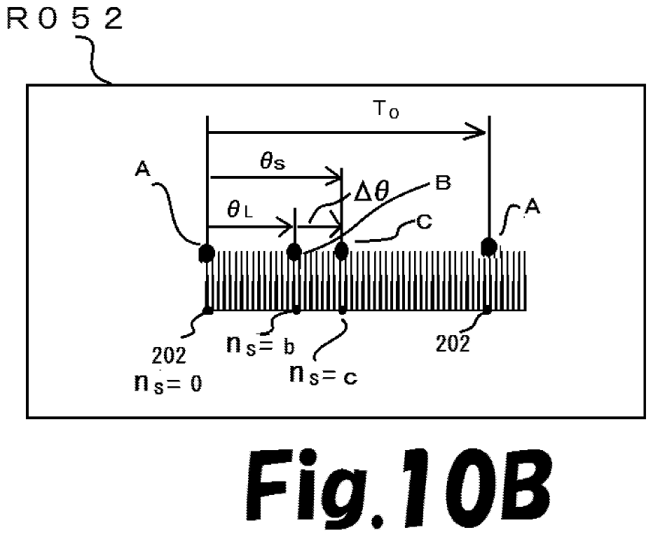

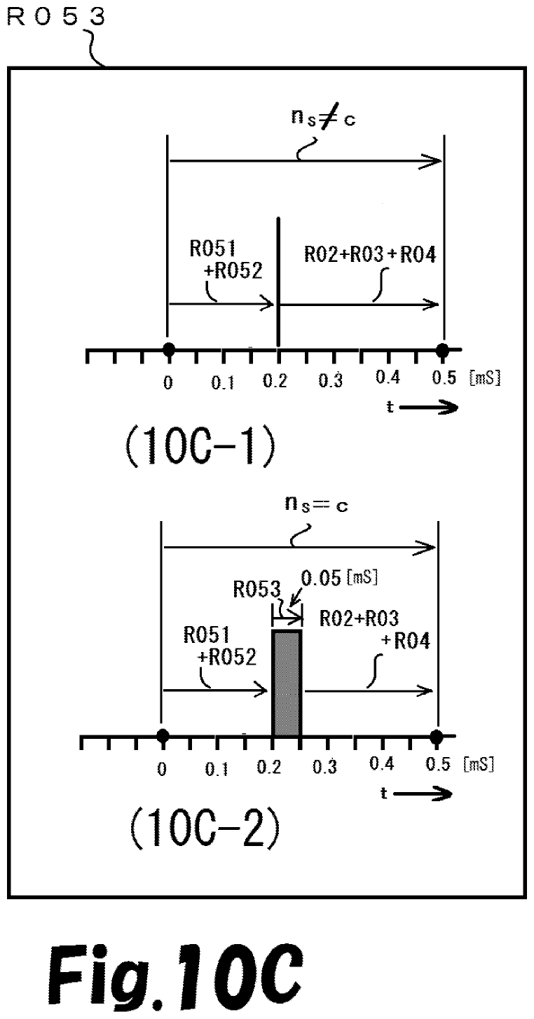

In routine R051, the LCD sunglasses 10 are controlled according to the procedure illustrated in FIG. 10A.

In routine R0510, the fundamental waveform WF specified by the control parameters is selected from among the various fundamental waveforms WF described in FIG. 3, and values such as the upper and lower limit values or the amplitude, phase, and coefficients are selected to create a waveform pattern ranging from −1 to +1.

In routine R0512, values are converted so that the transmittance target value 205, which is the output of the transmittance target generator 207 described in FIG. 2, is outputted in the range from 0% to 100%.

In routine R0514, the approximation formula described in FIG. 5A is used to compute the absolute value 240 of applied voltage from the inverse mapping correction part 237.

In routine R0516, the voltage value of the absolute value 240 of applied voltage is outputted from the analog output terminal A01 illustrated in FIG. 6.

In routine R0518, the polarity reversal signal 255 is switched on the frequency F2 and outputted from the digital output terminal DO1 illustrated in FIG. 6. Specifically, a square wave taking the logical value “high” or “low” depending on the numerical value of the loop number ns is outputted.

As a result, the AC voltage 260 is generated in the polarity reversal part 250 described in FIG. 5B, and is applied to the LCD element 60.

FIG. 10B is a diagram explaining a process related to the phase difference Δθ applied in routine R052, and schematically illustrating the timing of the sampling control that divides the cycle T0 of the light stimulation 200 into sampling intervals and repeats for each loop number ns.

Point A as the start point 202 of the control cycle T0 is located at the loop number ns=0.

Point B exists at the location moved from point A by the light stimulation onset delay θL, and let ns=b be the loop number at point B.

The numerical value of θL from points A to B is measured in advance using the apparatus in FIG. 6 on the basis of the waveform pattern for controlling the transmittance of the LCD element 60. The phase difference Δθ between the light stimulation 200 and the sound stimulation 210 is assigned a desired value based on clinical trial results.

Thereafter, the relation θs=θL+Δθ is used to compute point C, which corresponds to the timing θs for generating the sound stimulation 210. In other words, let ns=c be the loop number of point C at which the phase angle from the start point corresponds to θs, assuming a phase angle of 360 degrees from the start point to the end point of the cycle T0.

FIG. 10C illustrates control of the earphones 168 in routine R053.

The time series chart (10C-1) is the process if the loop number ns is not c. In this case, R02, R03, and R04 are performed immediately after R051 and R052. In other words, R053 is omitted.

The time series chart (10C-2) is the process if the loop number ns is c. In this case, R053 is performed immediately after R051 and R052, and then R02, R03, and R04 are performed.

In R053, the digital output terminal D03 in FIG. 6 is raised to the logical value “high”, and then the logical value “low” is outputted after waiting for 50 microseconds (0.05 ms) in this example. With this arrangement, the sound stimulation 210 is generated with a pulse width of 50 microseconds and frequency components that span the entire audible frequency range from Δθ Hz to around 20 kHz. Note that when driving the earphones 168 in FIG. 6 with the audio amplifier, a frequency filter may be added to adjust the frequency characteristics of the pulsed sound to adjust the timbre (that is, the pattern of the frequency characteristics curve) so that the amplitude of each of the frequency components in the sound are equal. Note that the timbre of the sound stimulation 210 also changes if the pulse width is changed in R053.

### Additional Implementation Details

This concludes the detailed description of the preferred embodiments.

Hereinafter, several alternative embodiments for achieving the present invention will be described.

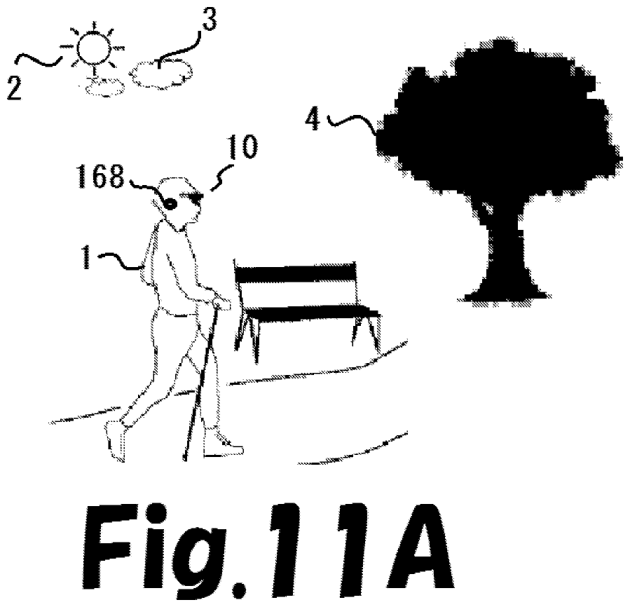

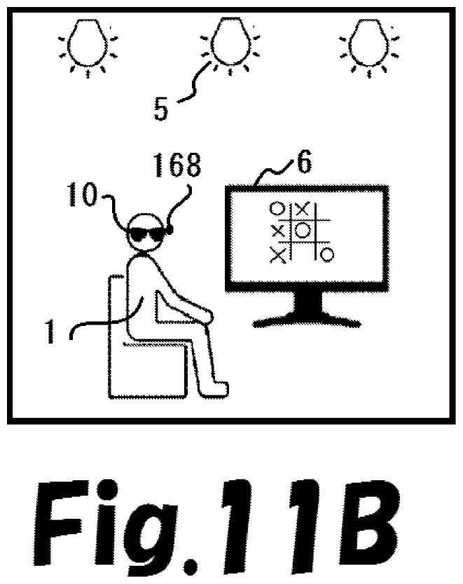

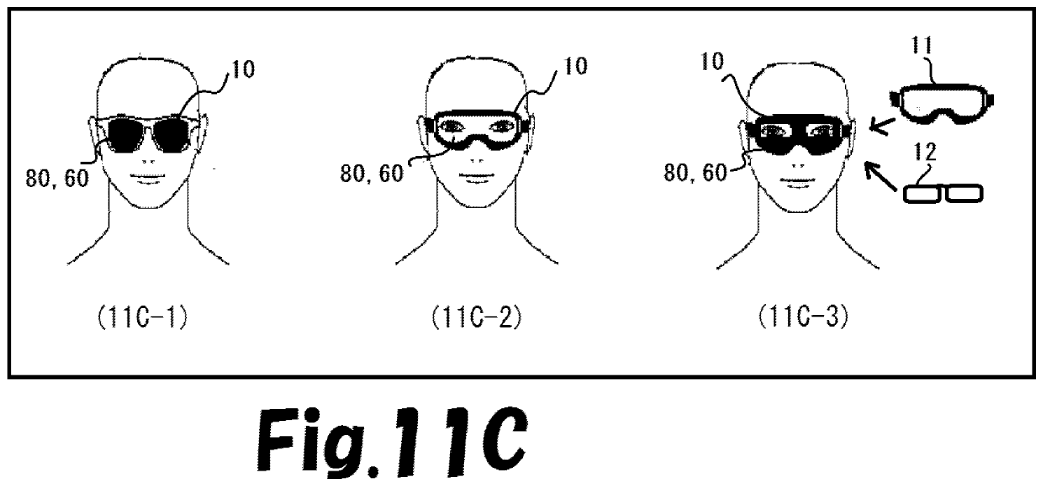

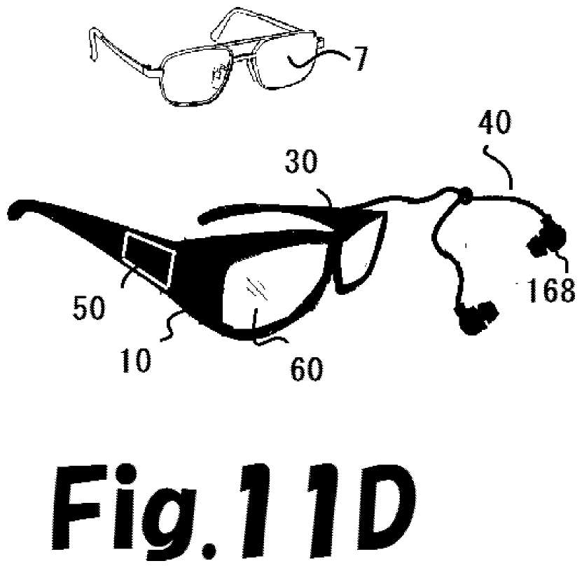

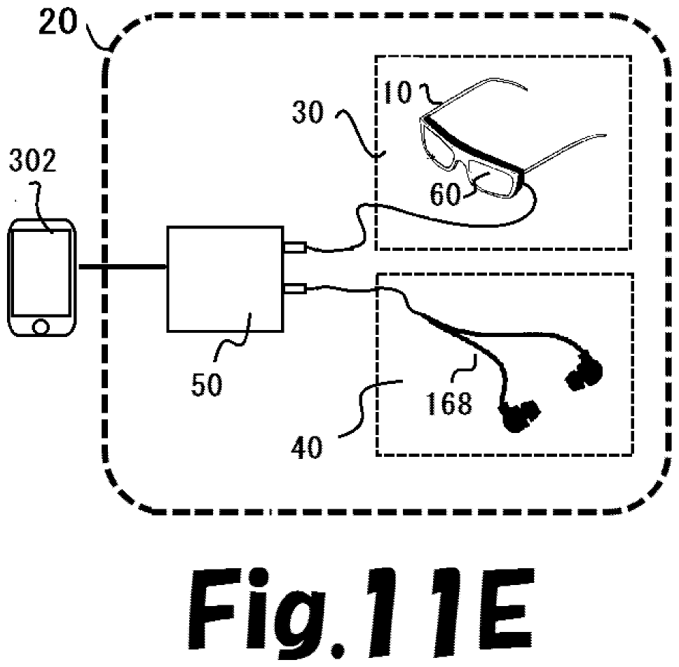

FIG. 11A illustrates a subject 1 walking through an outdoor park or a rehabilitation garden in a hospital as the dazzling light of the sun 2 is frequently blocked by clouds 3 and shade 4. The LCD sunglasses 10 and the earphones 168 as portable therapy devices are worn on the head. To prevent traffic accidents with automobiles, motorcycles, and the like when performing exercise therapy outdoors, as an alternative embodiment, it is desirable to use bone conduction earphones to hear ambient sound instead of the earphones 168. However, if bone conduction earphones are used, there is a possibility that the volume and frequency characteristics of the sound stimulation will be degraded. Note that for the sound stimulation 210, sufficient clinical trials should be conducted to determine what degree of volume and what frequency range and timbre of sound are appropriate.

FIG. 11B illustrates a subject 1 using a cognitive therapy game on a display 6 for a PC under ceiling lighting 5. In this diagram, the LCD sunglasses 10 and the earphones 168 as portable therapy devices are worn on the head, but as an alternative embodiment, a speaker may be used instead of the earphones 168 illustrated in the diagram. However, if the distance from the speaker to the subject 1 is long, the phase difference Δθ will increase and approach the warning level of 90 degrees as the sound stimulation 210 propagates through the air, which is not preferable. It is preferable to use, as much as possible, a sound-emitting device 160 that does not produce a propagation delay in the sound stimulation 210, such as wired earphones 168 or headphones.

FIG. 11C illustrates alternative embodiments of various outward shapes of the LCD sunglasses 10.

In the illustration (11C-1), the LCD element 60 is incorporated at the position of each of the left and right lenses 80 of 2 eyes glasses, and electrodes that provide the applied voltage to the two left and right LCD elements are electrically connected in parallel with each other. Outwardly, a variety of designs are possible, such as glasses, sunglasses, or 2 eyes goggles for competitive swimming.

In the illustration (11C-2), a single wide LCD element is incorporated instead of a transparent plate as the lens 80 of 1 eye goggle, and designs such as protective goggle for painting or toxic work, underwater glass, or sports goggle such as for skiing and motorcycling are possible.

In the illustration (11C-3), 2 eyes LCD sunglasses 12 are incorporated into work goggle 11 as an example of the LCD sunglasses 10 for clinical use that are shielded by metal foil so that light and electromagnetic fields do not enter from the front and sides. A separate audio-visual control device 50 with sufficient electromagnetic noise countermeasures is connected with sufficient electrical grounding and strictly shielded cables to prevent electrostatic induction as well. The brain waves of the subject 1 were measured during entrainment treatment using this device with electromagnetic noise countermeasures, and when light stimulation was generated at a frequency of Δθ Hz, for example, the noise in that frequency component was suppressed to less than +0.1 microvolts.

An experiment was also conducted to compare “an experimental group subjected to a cognitive load of singing while reading difficult lyrics during light and sound entrainment treatment” to “a control group that was subjected to a cognitive load without entrainment”. As a result, the phenomenon of an amplitude increase of a few microvolts in Δθ Hz brain waves was observed in the experimental group. The phenomenon of an increase in coherence between EEG electrodes was also observed. Both phenomena could be detected at a statistically significant level by verifying the data measured by the electroencephalograph in MATLAB (R).

In other words, the experiment confirms that the sensory stimulation therapy device 20 using the LCD sunglasses 10 of the present invention is effective in improving functional connectivity because a cognitive load can be imposed while the subject looks at an object with their eyes, and at the same time, coherence increased significantly in the experimental group subjected to entrainment.

FIG. 11D is a product concept of oversize LCD sunglasses 10 that can be used while the subject 1 wears glasses 7 for daily use, with the audio-visual control device 50 and the earphones 168 built in. Obviously, the earphones 168 may also be replaceable. The temple portions of the glasses may also be reinforced and tiny speakers may be mounted in place of the earphones 168.

FIG. 11E is a conceptual diagram in which the sensory stimulation therapy device 20, which comprises the visual stimulator 30, the auditory stimulator 40, and the audio-visual control device 50, is connected to the smartphone 302 in a wired or wireless manner. Since an example of remote control over a wireless connection using Wi-Fi is described in FIG. 9A, the following describes an alternative embodiment involving a wired connection.

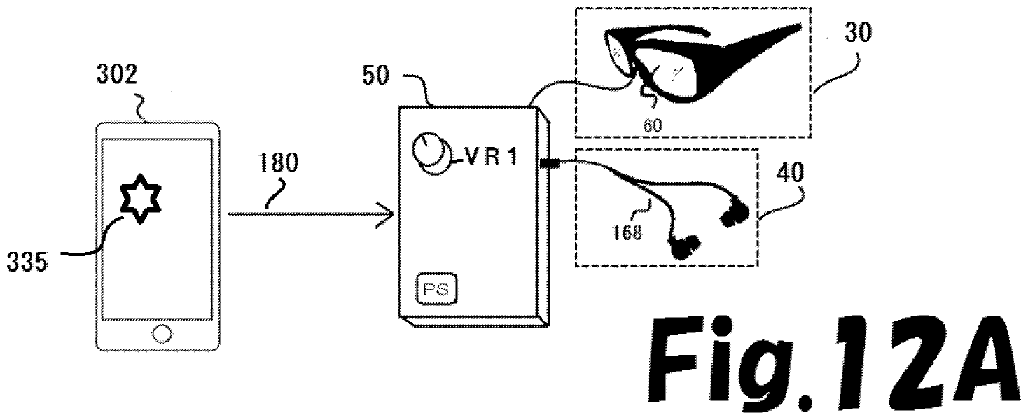

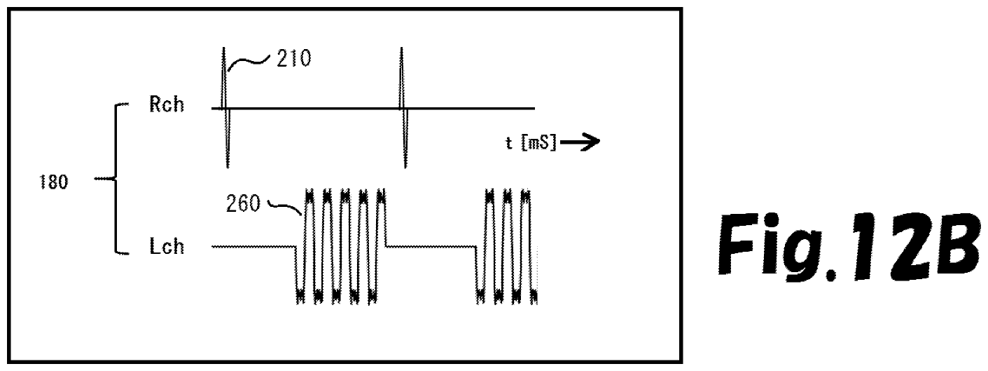

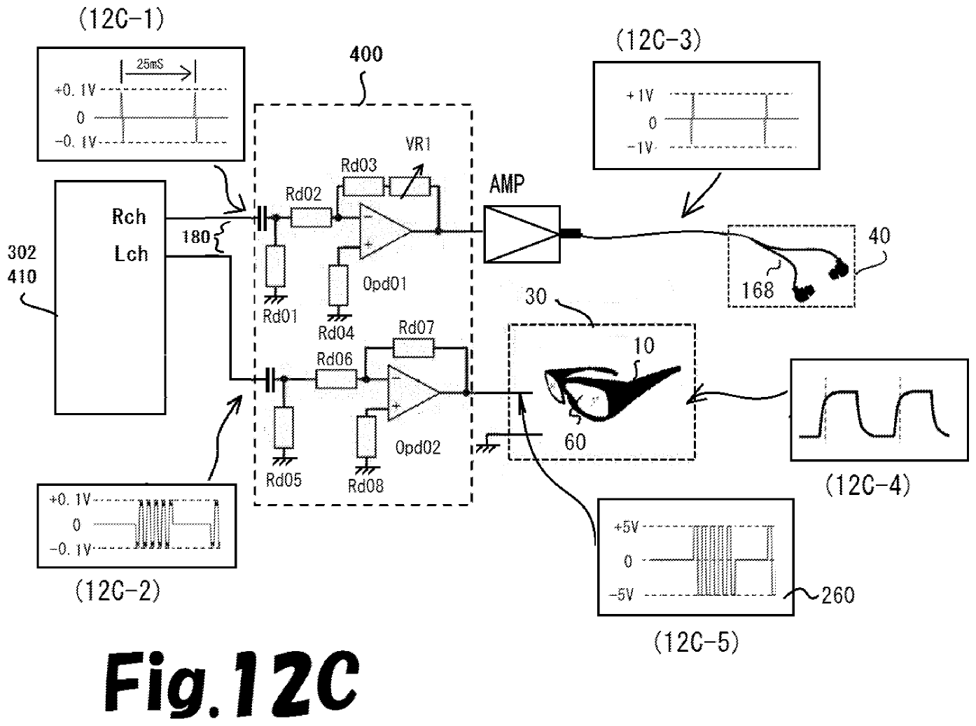

FIG. 12A is an alternative embodiment that uses the smartphone 302 as a music file playback device to transmit phase-synchronized light stimulation 200 and sound stimulation 210 data from a stereo audio output 180 by wire to the audio-visual control device 50.

For example, audio data of sound stimulation 210 as a click sound with a pulse width of 50 microseconds is outputted to the right channel Rch in FIG. 12B. A case is illustrated in which the waveform of the AC voltage 260 generated by reversing the polarity in the polarity reversal part 250 on the basis of the transmittance target value 205 with a 50% duty cycle illustrated in the time series graph (7-2) in FIG. 7 is outputted to the left channel Lch. This is merely one example given for the sake of illustration.

The important point in this alternative embodiment using the stereo audio output 180 is that the waveform patterns of the light stimulation 200 and the sound stimulation 210 and the phase difference Δθ are a stereo signal, and thus are phase-synchronized in advance. Consequently, the audio-visual control device 50 that demodulates the stereo audio output 180 has an extremely simple and low-cost structure.

FIG. 12C is a diagram explaining the structure of the audio-visual control device 50 that demodulates the stereo audio output 180.

The functions of the smartphone 302 are achieved with a music file playback device 410, for which a music CD, a PC, or a dedicated music playback device may be used. Any of various known formats, such as MP4, MP3, and AVI, may be used as the format of the music file.

The time series chart (12C-1) illustrates the output voltage of the right channel Rch. The time series chart (12C-2) illustrates the output voltage of the left channel Lch.

A demodulator 400 forms a major portion of the audio-visual control device 50, and amplifies the input voltage of the right channel Rch while adjusting the volume with a volume VR1 for playback as a drive signal 45 through an audio amplifier AMP and output to the earphones 168 as the sound-emitting device 160.

A waveform of +0.1 V inputted from the left channel Lch is amplified from 50 to 100 times until reaching the AC voltage 260 with a waveform that saturates at +5 V in the time series chart (12C-5), and is applied to the LCD sunglasses 10.

The time series chart (12C-4) is the waveform pattern of the specific transmittance P generated by the LCD sunglasses 10.

If multiple types of stereo signal music files containing information on the waveform patterns of the light stimulation 200 and the sound stimulation 210 and the phase difference Δθ are created for each waveform pattern, the user can conveniently switch the waveform patterns and phase difference Δθ of the light and sound entrainment to be used for treatment all at once by simply switching the music file to be played back.

To generate a stereo signal music file containing information on the waveform patterns of the light stimulation 200 and the sound stimulation 210 and the phase difference Δθ, the waveform patterns of the light stimulation 200 and the sound stimulation 210 may be generated with the phase difference Δθ by the development apparatus in FIG. 6, and the electrical signals may be recorded with a data logger capable of high-speed sampling for music.

Alternatively, control operations can be simulated in MATLAB (R) to generate a stereo signal music file approximately 10 seconds long in which the waveform patterns of the light stimulation 200 and the sound stimulation 210 are phase-synchronized with the phase difference Δθ, and the music file can be edited using music data editing software to complete a 30-minute or 1-hour music file for treatment.

In other words, the audio-visual control device 50 accepts the input of a stereo signal 180 obtained by playing back a “stereo sound file 220 in which the light stimulation 200 and the sound stimulation 210 are each recorded in separate channels with the phase difference Δθ”, and outputs the stereo signal 180 to the visual stimulator 30 and the auditory stimulator 40, wherein for the visual stimulator 30, the stereo signal is demodulated into the applied voltage in which the polarity of the absolute value 240 of the applied voltage calculated from the transmittance target value 205 of the LCD element 60 is reversed at the reversal frequency F2, and applied to the LCD element 60, and for the auditory stimulator 40, the stereo signal is played back as the drive signal 45 and supplied to the sound-emitting device 160.

Incidentally, when generating stereo music files by simulation, not only click sounds but also chirp sounds widely used in ASSR can be generated using the chirp function in MATLAB (R) and used as the sound stimulation 210. In other words, the waveform pattern of the sound stimulation 210 is a click sound and/or a chirp sound.

This disclosure of the invention is the product of an early pilot study, prior to clinical research trials. With this disclosure, the device can be produced as “equipment for clinical research”, and a clinical research tool can be provided to researchers with a medical background.

However, it should be noted that this device is a medical device that “poses a risk to the human body in the event of a malfunction”. Therefore, when this device is used in clinical research involving human subjects, it is a prerequisite that the person in charge of the clinical research must decide not only whether to use the device, but also the conditions for conducting clinical research, such as control parameters and subject selection when the device is to be used, on the basis of their own safety assurance and medical judgment.

The foregoing description of preferred embodiments of the present invention is presented for illustrative and explanatory purposes. The present invention is not exhausted by or limited to the precise forms disclosed. Numerous modifications and variants are possible in the light of the above teachings. The scope of the present invention is to be limited not by the detailed description but by the claims attached to this specification. The present invention resides in the claims because numerous embodiments of the present invention are possible without departing from the spirit and scope thereof.

What is claimed is:

1. A sensory stimulation therapy device using LCD sunglasses for preventing and treating neurological disorders involving impairment of functional connectivity of brain and provided with a structure enabling a subject of treatment to visually perceive a surrounding environment during treatment action, wherein
the sensory stimulation therapy device comprises a visual stimulator, an auditory stimulator, and an audio-visual control device,
the visual stimulator is LCD sunglasses that generate light stimulation by controlling transmittance of light incident from the surrounding environment transmitted through an LCD element by placing the LCD element as a lens of 2 eyes or wide 1 eye LCD sunglasses,
the auditory stimulator is a sound-emitting device that generates sound stimulation on an audible frequency, and
the audio-visual control device is control means that generates the light stimulation by supplying an applied voltage to the LCD element, generates the sound stimulation by supplying a drive signal to the sound-emitting device, and controls a phase difference Δθ from the light stimulation to the sound stimulation,
sets, as a timing B representative of a phase of the light stimulation, an instant of zero-crossing as a fundamental frequency component of a waveform pattern of the transmittance repeatedly generated by the LCD element increases, and
supplies the drive signal to the sound-emitting device at a timing C shifted from the timing B by the phase difference Δθ.

2. The sensory stimulation therapy device using LCD sunglasses of claim 1, wherein an absolute value of the phase difference Δθ is less than ¼ of one cycle of the light stimulation.

3. The sensory stimulation therapy device using LCD sunglasses of claim 1, wherein the audio-visual control device calculates an absolute value of the applied voltage from a transmittance target value of the transmittance of the LCD element and applies, to the LCD element, an AC voltage with a polarity of the absolute value of the applied voltage reversed at a reversal frequency F2.

4. The sensory stimulation therapy device using LCD sunglasses of claim 1, wherein the audio-visual control device uses a variable with a data structure expressing two or more numerical value levels as the transmittance target value of the LCD element and/or the absolute value of the applied voltage.

5. The sensory stimulation therapy device using LCD sunglasses of claim 4, wherein the audio-visual control device describes a control target of the transmittance of the LCD element as a time function of time containing one fundamental frequency.

6. The sensory stimulation therapy device using LCD sunglasses of claim 4, wherein the audio-visual control device describes a control target of the transmittance of the LCD element as a time function containing multiple fundamental frequencies.

7. The sensory stimulation therapy device using LCD sunglasses of claim 4, wherein the audio-visual control device describes a control target of the transmittance of the LCD element as a periodic waveform pattern with a defined value at each sampling time.

8. The sensory stimulation therapy device using LCD sunglasses of claim 1, wherein
the audio-visual control device accepts input of a stereo signal obtained by playing back a stereo sound file in which the light stimulation and the sound stimulation are each recorded in separate channels with the phase difference Δθ, and outputs the stereo signal to the visual stimulator and the auditory stimulator,
for the visual stimulator, the stereo signal is demodulated into the applied voltage with the polarity of the absolute value of the applied voltage calculated from the transmittance target value of the LCD element is reversed at the reversal frequency, and applied to the LCD element, and
for the auditory stimulator, the stereo signal is played back as the drive signal and supplied to the sound-emitting device.

9. The sensory stimulation therapy device using LCD sunglasses of claim 8, wherein the waveform pattern of the sound stimulation is a click sound and/or a chirp sound.
